(19) United States
(12) Patent Application Publication
(10) Pub. No.: US 2025/0377333 A1
(43) Pub. Date: Dec. 11, 2025

(54) ELECTROPHORETIC METHOD, DEVICES AND SYSTEMS

(71) Applicant: University of Tasmania, Sandy Bay, Tasmania (AU)

(72) Inventors: Arushi Manchanda, Sandy Bay, Tasmania (AU); Vipul Gupta, Sandy Bay, Tasmania (AU); Brett Paull, Sandy Bay, Tasmania (AU)

(21) Appl. No.: 19/124,756

(22) PCT Filed: Oct. 27, 2023
(86) PCT No.: PCT/AU2023/051091

§ 371 Date: Apr. 25, 2025

(30) Foreign Application Priority Data
Oct. 28, 2022 (AU) 2022903209

(51) Int. Cl. G01N 27/447

(52) U.S. Cl. CPC G01N 27/44743; G01N 27/44756

(57) ABSTRACT

The present disclosure relates to a method of separation of charged substances by electrophoresis by using an inorganic thread. It includes contacting an electrolyte with an outer surface of the inorganic thread to cause wetting the outer surface of the inorganic thread and wicking along the inorganic thread, loading the charged substances into the electrolyte wetting the outer surface of the inorganic thread in the region of a loading zone portion of the inorganic thread and applying an electric field across the length of inorganic thread to cause the charged substances to be separated along the inorganic thread under the influence of the electric field. The present disclosure also relates to a method for the electrophoretic transfer of a charged substance from a sample on a sample applicator to an inorganic thread and to a method of stripping an outer surface coating from an inorganic thread having an outer surface coating. Related systems and components are also disclosed.

### RELATED APPLICATION

The present application claims the benefit of Australian provisional patent application No. 2022903209 filed on 28 Oct. 2022, which is incorporated herein by reference in its entirety.

### TECHNICAL FIELD

The present disclosure relates to methods, devices and systems for the separation of charged substances by electrophoresis. The present disclosure also relates to methods and systems for the transfer of samples collected on swabs (or similar sample applicators) to particular types of substrates for subsequent analysis or other processing. Such transfers are particularly relevant to collection and concentration of charged components in a sample by electrophoresis.

### BACKGROUND OF THE DISCLOSURE

The ability to collect and concentrate charged substances at low concentrations is critical in a multitude of scenarios, including the development of improved point-of-care (POC) testing useful in biochemical, pharmaceutical, forensic, environmental and other analytical procedures. Recent advances in POC testing mean that testing devices are now low cost, easy to use, and allow the ability to collect both liquid and dry samples from a variety of locations. This avoids the delays of having to transport the samples back and forth to a laboratory setting, hampering timely analysis and decision making, especially in life threatening circumstances, such as disease diagnosis, terrorism threats, epidemics, or pandemics, and so forth.

Traditionally, POC analytical devices have been developed using microchannels as fluidic conduits. More recently, other substrates such as paper and textiles have been considered for providing fluidic conduits. Whilst those alternatives have been studied to some extent, microfluidic textile analytical devices (μTADs) have a number of deficiencies. One problem with such devices is the fact that the paper or textile substrates are based on organic materials, and therefore the sample that has been separated/concentrated on the device must be taken off the substrate to be capable of detection. However, to avoid such issues with the paper and textile substrates requires a return to microfluidic conduits that suffer from problems with the regulation of fluid flow, capillary pressure and the fact that the conduits are “closed systems” that do not enable access to the sample mid-way along the conduit. Such microfluidic devices also tend to be technically challenging and therefore also costly to produce.

Capillary electrophoresis (CE) is a separation technique that enables sample separations to be performed at low concentration and low sample volumes and gives improved resolution of samples as compared to high-performance liquid chromatography. However, the use of CE in detection of low concentrations in a capillary comes with its own inherent problems, such as optical distortion due to the curvature of the capillary walls. This is particularly the case when utilising UV-Vis absorbance.

Hence, an object of some embodiments of the present disclosure is to develop a different way to separate charged substances to enable subsequent detection of the charged analytes or biomolecules in low concentration samples.

It has also been recognised that it would be advantageous for the system to be able to perform multiple parallel runs, to achieve multiplexed or high throughput analysis. Providing such functionality would minimise the average sample analysis time in situations where large numbers of samples are being taken for laboratory analysis, such as epidemics and pandemics.

Another object for some embodiments of the present disclosure relates to the provision of a simple technique for transferring a sample to the separation device in a manner that allows for good sample transfer without excessive “dilution” of the sample. Such factors are important when low sample concentrations are involved, and a diagnostic result is desired within a short time-period.

Owing to the widespread use of microfluidic devices in disease diagnosis, forensic investigations, threat analysis, pharmaceutical analysis, environmental analysis and laboratory-based chemical analyses and so forth, the developed technology is of utility in various applications.

### SUMMARY OF THE DISCLOSURE

According to a first aspect, the present disclosure provides a method for separating charged substances by electrophoresis, comprising:
providing a length of an inorganic thread;
contacting an electrolyte with an outer surface of the inorganic thread so as to cause wetting of the outer surface of the inorganic thread,
loading the charged substances into the electrolyte wetting the outer surface of the inorganic thread in the region of a loading zone portion of the inorganic thread;
applying an electric field across the length of inorganic thread to cause the charged substances to be separated along the inorganic thread under the influence of the electric field.

In the wetting step, the contacting of the electrolyte with the outer surface of the inorganic thread causes wetting of the outer surface of the inorganic thread and wicking along the inorganic thread.

Whereas prior art microfluidic textile devices have relied on organic materials—such as nylon, cotton, polymeric compounds and similar, to be used for the textile component, the present applicant has surprisingly found that a corresponding device can be produced using an inorganic thread—such as a silica thread or otherwise.

The prior art devices rely on some absorption and wicking of the electrolyte along the textile or paper material. However, the applicant has conducted test work in order to show that wetting of inorganic threads can be achieved in a similar manner, and this enables suitable devices to be produced. The inorganic thread enables the required fluid flow through wicking occurring between the filaments or fibrils that make up the thread. The applicant has also conducted experiments in order to show that wetting and wicking between the filaments or fibrils that make up a thread assists transfer of an analyte from, e.g., a swab to the inorganic thread. The applicant has further shown that inorganic threads achieve suitable wetting in that the inorganic threads retain fluid, e.g., electrolyte or buffer.

Another advantage that flows from the use of the inorganic thread material is the absence of a fluorescence background. In this respect, the inorganic thread is effectively “transparent”. This differs from organic threads, such as nylon and other polymers. The applicant has conducted test work showing that an inorganic thread provides a higher fluorescence signal-to-noise ratio than a conventional thread, such as nylon and other polymers, even at low analyte concentrations.

A further advantage is the ability to modify the outer surface of the inorganic thread through reacting the outer surface groups with suitable reagents to modify the inorganic thread outer surface in any way that is desired.

Also, as compared to silica capillaries, the inorganic thread is “open” to the environment, so the sample can be loaded at any point as required along the thread and can be accessed or contacted as required at any point downstream from where it is loaded along the thread. This allows a section of the thread which has already been used to separate a charged compound from a substance to be removed and transferred to, e.g., a pathology laboratory as a dry sample. The inorganic thread enables the required fluid flow along the thread in the manner of a type of capillary, through wicking occurring between the filaments or fibrils that make up the thread. The device in some ways may be thought of as an “inverted capillary” or “inverted column” where the liquid flow is on the outside rather than through a central capillary aperture. This also contrasts to fluid flow through the central channel portion of a microfluidic microchannel.

The applicant has also found that the configuration of the inorganic material is important. The applicant has conducted test work which shows that the performance of an inorganic thread surpasses that of an inorganic fabric. An analyte on an inorganic fabric (e.g., woven silica strips) is more difficult to load, reliably separate, focus and recover owing to multi-directional diffusion in the fabric, which also requires lower voltage to be used and less sample to be loaded.

Inorganic threads also tend to be more stable (i.e., less prone to degradation or spoilage) than organic threads and contain less impurities in the inorganic material than the impurities content often found in organic threads. Inorganic threads may thus provide for more reliable, accurate and repeatable analyses than organic threads.

All the above factors combine to yield a useful method and corresponding device for performing separations of charged substances based on low concentrations. In a second aspect, there is also provided an electrophoresis device for separating charged substances by electrophoresis, comprising:
a length of inorganic thread with an outer surface that is wettable by an electrolyte; and
means for applying an electric field along the length of inorganic thread, so that when charged substances are loaded onto the inorganic thread, the charged substances are separated along the inorganic thread under the influence of the electric field.

The inorganic thread allows wicking, as described above.

In commercial operations, it is possible to provide an electrophoresis instrument that includes the machinery and analytical components (e.g., detector, electronic components and computer processing), and to separately provide a disposable cartridge for loading into or onto the apparatus. The cartridge may comprise the components of a first electrolyte reservoir, a second electrolyte reservoir, and the inorganic thread extending between the first and second electrolyte reservoirs onto which a charged substance can be loaded. The cartridge may additionally comprise first and second electrodes, otherwise the electrophoresis instrument may comprise those components, which may then be inserted into the electrolyte reservoirs upon loading of the cartridge into the apparatus, or prior to performance of the separation process. The cartridge may comprise electrolyte, either in the electrolyte reservoirs or otherwise. In the alternative, electrolyte may be dispensed into the electrolyte reservoirs prior to performance of the separation process.

Following from the above, in a third aspect, the present disclosure provides a cartridge for use in an electrophoresis instrument, the cartridge comprising:
a first electrolyte reservoir;
a second electrolyte reservoir;
an inorganic thread extending between the first and second electrolyte reservoirs onto which a charged substance can be loaded.

In a separate but related concept, in accordance with a fourth aspect, the present disclosure also provides a method for the electrophoretic transfer of a charged substance from a sample on a sample applicator to an inorganic thread, the method comprising:
contacting the sample applicator containing a sample including the charged substance with the inorganic thread in the presence of an electrolyte, or
immersing the sample applicator containing a sample including the charged substance into an electrolyte solution, and contacting the electrolyte solution with the inorganic thread; and
applying an electric field along the inorganic thread to effect transfer of the charged substance from the sample applicator and/or the electrolyte solution to the inorganic thread.

In the fourth aspect, there is provided a simple manner for transferring a sample onto the inorganic thread. The inorganic thread may be viewed as a form of “electrophoresis matrix” on which the electrophoretic separation (and concentration) of charged substances is performed, for the separation of charged substances into bands, which allows for a subsequent operation to be performed on the charged substance. The fact that a sample applicator (e.g., a swab, bud, wad, wipe, sampling swiper or otherwise) can be wiped across a surface and the charged substances transferred to the inorganic thread at a high transfer rate (i.e., high % of substances transferring over to the inorganic thread) is a notable feature. The sample applicator may be placed in contact with the thread or immersed in an electrolyte solution with which the thread is contacted, and the charged substances transferred to the inorganic thread at a high transfer rate. In either scenario, transfer of the charged substances to the inorganic thread is achieved through the action of applying a voltage potential across the thread (via a pair of electrodes), in the presence of an electrolyte, which results in a high transmission of the charged substances from the sample applicator onto the inorganic thread. This is achieved for the first time herein using an inorganic thread as the electrophoretic matrix. This results in a form of concentration of the charged substance onto the thread. Concentration is achieved without a selective membrane positioned between the sample applicator and the electrophoresis matrix (i.e., the inorganic thread).

The inorganic thread as the electrophoresis matrix is in contact with electrolyte during the application of the electric field. For example, the electrolyte may comprise a volume of the electrolyte—i.e., the “bulk electrolyte” that wets the inorganic thread, or the electrolyte may wet the electrophoresis matrix by coating or wicking of the inorganic thread by the electrolyte. The applicant has surprisingly found that, where the inorganic thread as the electrophoresis matrix is in a bulk electrolyte, rather than diffusing into the bulk electrolyte, the charged substance follows the pathway of the electric field and transfers from the sample applicator directly to the inorganic thread as the electrophoresis matrix. Wetting of the inorganic thread by the electrolyte in the absence of a volume of bulk electrolyte similarly enables the transfer of a high percentage of the charged substance from the sample applicator onto the inorganic thread in a concentrated zone. Thereafter, the charged substance can be further processed or moved along the inorganic thread as desired. Examples of options for further processing or transferring the charged substance along the inorganic thread are described herein.

According to a fifth aspect, the present disclosure further provides a system for the transfer of a charged substance from a sample on a sample applicator to an inorganic thread, the system comprising components including:
a sample transfer reservoir;
an inorganic thread positioned through the sample transfer reservoir;
a pair of electrodes positioned to enable the application of an electric field across the inorganic thread;
   wherein the components are positioned such that the sample applicator contacts the inorganic thread during the application of an electric field along the inorganic thread to enable transfer of the charged substance from the sample applicator to the inorganic thread.

The system may further comprise a receiver for receiving the sample applicator (e.g., the swab). In some embodiments the sample transfer reservoir may serve as the receiver for receiving the sample applicator, or the receiver may be in the form of a separate feature of the device into which the swab is positioned, before it is moved into contact with the thread in the sample transfer reservoir.

It is also noted that the system or device may be in the form of a cartridge. Alternatively, the system may include a cartridge that provides one or more of the components of the system described above. Further details of this cartridge-type arrangement and other possible arrangements are described below.

The use of an inorganic thread as the electrophoretic matrix that creates a pathway for an electric field in an open system, such as a thread in particular, offers various advantages over conventionally used microchannels. These include high flexibility, high mechanical strength even under wet conditions, reusability, disposability, and ease of functionalisation and arrangement into complex 2D and 3D structures. Moreover, inorganic thread-based devices do not require pumping systems and allow easy manipulation and on-line modification of the sample. The on-line modification is due in part to the “open” nature of the inorganic thread. Specifically, the environment may be modified along the inorganic thread without restriction (e.g., another substance can be added, a sample taken, etc)—this contrasts to a “closed” capillary which is not open to the environment and cannot be modified in the same manner (e.g., another substance cannot be added into the channel without an access opening in the capillary). In addition, when used, the simple transfer of a swabbed sample to an inorganic thread electrophoresis system as described in the present disclosure, with a high degree of sample transfer and minimal loss, circumvents the need for swab transport and/or sample desorption into a solution, providing a low concentration sample solution.

The method of the present disclosure in some embodiments involves a simple step of placing the swab in contact with the inorganic thread either through a designated sample transfer reservoir or sample receiver of the analytical device, where a quantitative transfer, or near-quantitative transfer, of the charged substances (including potential analytes) is performed from the swab onto the thread. The transfer is achieved by simply bringing the swab and the inorganic thread into direct contact and applying a voltage potential across the thread. The test work presented herein indicates that close to 100% recovery of analytes can be achieved. These results can be achieved with a range of different types of analytes and a range of swabs (both dry and wetted by electrolyte). The degree of transfer may be at least 50%, 60%, 70%, 80% or at least 90% of the target charged substances from the sample applicator to the inorganic thread as the electrophoretic matrix. The transfer of the charged substance from the sample applicator to the inorganic thread as the electrophoretic matrix in some embodiments can occur to the substantial exclusion of uncharged substances in the sample. This is achieved by suppressing electro-osmotic flow, through which charged substances can be transferred to the inorganic thread and not the uncharged substances. The transferred analytes can also be successfully manipulated on the threads using procedures, such as isotachophoresis, electrophoresis, sample splitting, or physical movement of the thread itself. In other embodiments, where it is desired to transfer uncharged substances to the inorganic thread as the electrophoretic matrix in addition to charged analytes, it may be possible to modify the conditions to achieve this.

According to a further aspect, the present disclosure provides a system for the transfer of a charged substance from a sample on a sample applicator to an inorganic thread, the system comprising components including:
a sample transfer reservoir;
an inorganic thread positioned through the sample transfer reservoir;
a pair of electrodes positioned to enable the application of an electric field across the inorganic thread;
   wherein the components are positioned such that the sample applicator contacts the inorganic thread during the application of an electric field along the inorganic thread to enable transfer of the charged substance from the sample applicator to the inorganic thread.

In some embodiments, the system comprises a first electrolyte reservoir in which a first of the pair of electrodes is positioned, and a second electrolyte reservoir in which a second of the pair of electrodes is positioned, with the inorganic thread extending between the first and second electrolyte reservoirs, wherein the sample transfer reservoir is positioned between the first and second electrolyte reservoirs along the thread.

In some embodiments, the system comprises the following components:
a first electrolyte reservoir comprising a first electrode;
a second electrolyte reservoir comprising a second electrode;
an inorganic thread extending between the first and second electrolyte reservoirs;
a third reservoir through which the thread passes between the first and second electrolyte reservoirs which functions as the sample transfer reservoir; and
a controller for controlling the application of an electric field across the inorganic thread so as to effect movement of a charged substance along the inorganic thread in a direction towards the second electrolyte reservoir;
   wherein the third reservoir enables the charged substance to be loaded onto the thread in the third reservoir.

Alternatively, the third reservoir may function as an operation reservoir where an operation is performed on the charged substance transferred to the third reservoir following movement of the charged substance along the thread on the application of the electric field.

The applicant has devised this arrangement comprising at least three reservoirs—including separate reservoirs for the first and second electrodes and at least one other reservoir, which may be an intermediate reservoir (the third reservoir) positioned along the inorganic thread between the first and second electrode-containing reservoirs. The third reservoir is free of any electrode. There may be one or more additional reservoirs in addition to the third reservoir. The additional reservoirs may be positioned along the inorganic thread between the first and second electrode-containing reservoirs. In alternative embodiments, the additional reservoirs (or some of these reservoirs) may be positioned before or after the first and second electrode-containing reservoirs. The application of an electric field between the first and second electrodes results in the application of an unbroken electric field across the inorganic thread extending through the third reservoir. If the third reservoir is positioned before or after the first and second reservoirs, sufficient electrophoretic conditions would be required to migrate the charged substances from that reservoir towards the first (and second) reservoirs.

In cases where system contains only three reservoirs, the sample may be loaded onto the inorganic thread in the first reservoir, and then the charged substance can be transported under the influence of the electric field along the thread towards and into the third reservoir. The charged substance may then be desorbed from the thread and into the bulk electrolyte in the third reservoir, where an operation may be performed. “Operation” refers to a chemical analysis, detection, coupling or modification of the charged substance. Examples include analyte detection, analyte modification, coupling of the charged substance to a marker, a chemical reaction or a transformation involving the charged substance, complex detection involving the charged substance (e.g., PCR) and so forth. This system provides flexibility in terms of the functionality of the system and the ability to perform operations in a liquid state, within a bulk electrolyte, rather than in the solid state or otherwise.

In an alternative arrangement for the three-reservoir system, the sample may be loaded onto the inorganic thread in the third reservoir, and then passed along the inorganic thread through electrophoresis. The charged substance that is moved along the inorganic thread between the third reservoir and the second electrolyte reservoir through the application of the electric field may then be used in any suitable process or subjected to any desired process. As one example, a zone of the thread following the application of the electric field may be cut away, and the cut portion subjected to further processing to recover the charged substance. Alternatively, an operation can be performed on the charged substance either on the inorganic thread or once it has been desorbed from the thread, either within a reservoir of the system, or otherwise.

In use, the reservoirs may contain electrolyte, and the inorganic thread is wetted with electrolyte—which may even be in the form of a conductive substance such as a hydrogel to provide an electrical pathway along the inorganic thread between the reservoirs. Charged substances may be desorbed into the electrolyte in particular reservoirs, as required by the process being undertaken. The system described herein allows for multiple operations or processes to be performed in multiple reservoirs, using an inorganic thread-and-reservoir arrangement, and the application of an electric field to transfer charged substance(s) between reservoirs. The charged substances can be desorbed from the inorganic thread into the bulk electrolyte, and re-loaded onto the inorganic thread as required. In the past, capillaries have been considered for moving substances from one bulk electrolyte to another. However, the present system provides flexibility in terms of providing the option to either retain the charged substance on the thread (concentrated), or to desorb into a bulk solution.

The third reservoir (and each additional reservoir) is free of any electrode, while still maintaining the electric circuit between the electrode carrying reservoirs. Where there are dedicated sample loading reservoirs and operation reservoirs, each of these reservoirs is free of any electrode.

In some embodiments, the system includes an array comprising multiple sets of said first and second electrolyte reservoirs, first and second electrodes, inorganic thread, and third reservoirs (and optionally any further reservoirs). If provided in cartridge form, each cartridge may be for a single set, or a single cartridge may contain multiple sets of the reservoirs, electrodes and inorganic thread. In an alternative arrangement, the cartridge may comprise first and second electrolyte reservoirs, with multiple inorganic threads spanning between the first and second electrolyte reservoirs, each inorganic thread including one or more intermediate reservoirs along its length. This may be referred to as a “thread splitting” arrangement. In this case, each thread shares the first and second electrolyte reservoirs with other inorganic threads, but each has its own sample loading reservoir(s) and/or sample operation reservoir(s) or zones.

An array of such components allows for multiple parallel runs to be performed to achieve multiplexed or high throughput analysis. These may be performed on either a single sample (or single sample applicator/swab) or from multiple samples (sample applicators/swabs). Multiplexed analysis can also be performed by splitting a single inorganic thread into multiple pathways, where each pathway was used to determine a specific marker to provide a more holistic sample analysis and minimise the false positive and negative results that are often obtained when a single marker is analysed. High throughput analysis can be performed by recruiting multiple threads, substantially in parallel, in which each thread is used to perform analysis on an individual swab, minimising the average sample analysis time in situations such as epidemics and pandemics.

Prior art multiplexed analytical devices tend to have been restricted to the use of electrode-coupled initial and terminating reservoirs. However, the above-described multiplexed configuration has been developed that allows the use of electrode-free reservoirs, facilitating multi-step analysis and minimising the risks of electrode fouling. In some embodiments, there may be one or more additional reservoirs arranged between the initial and terminating electrodes (the first and second electrodes) such that they do not break the electro-fluidic circuit while also allowing independent activities, such as sample introduction, concentration, modification, detection, selective uptake or release, etc. The developed system can facilitate the use of microfluidic (inorganic) “textile” analytical devices in performing complex analytical procedures, which are often required in real-world settings. By the term “textile” as used herein refers generally to fibre-based materials, including filaments, fibrils, threads, yarns, or an assembly thereof such as a fabric. Since microfluidic textile analytical devices use high voltages, the availability of electrode-free reservoirs for sample manipulation would also promote the generation of safer microfluidic textile analytical devices by preventing user exposure to the live electrodes. The present system, while suitably making use of a high voltage potential, uses low current and is designed for safe operation.

The voltage potential applied in some embodiments is at least 900 V. The current in some embodiments is less than 300 μA.

According to an additional aspect, there is provided a method for performing an operation on a charged substance, the method comprising:
providing a first electrolyte reservoir comprising a first electrode, a second electrolyte reservoir comprising a second electrode and an inorganic thread that extends between the first and second electrolyte reservoirs;
loading a charged substance onto the inorganic thread;
moving the charged substance along the inorganic thread to an operation reservoir positioned downstream in the flow direction of the charged substance, by applying an electric field across the inorganic thread between the first and second electrodes; and
performing an operation involving the charged substance in the operation reservoir.

In preferred embodiments the charged substance is taken from a sample, and is loaded onto the inorganic thread in a sample loading reservoir.

There may be more than one operation reservoir (or operation zones) traversed by the thread, and allowing for different operations to be performed in each of said reservoirs (or in each of said zones). The movement of the charged substance along the inorganic thread involves the application of an electric field across the thread.

According to yet another aspect, there is also provided a method of stripping an outer surface coating from an inorganic thread having an outer surface coating, comprising subjecting the inorganic thread to vacuum plasma treatment to strip the outer surface coating from the thread. There is also provided a treated inorganic thread when produced by this method.

### BRIEF DESCRIPTION OF THE DRAWING(S)

FIG. 1 is a schematic illustration of arrangements of a device comprising an inorganic thread in accordance with embodiments of the present disclosure. FIG. 1(a) is a schematic illustration of an arrangement with four reservoirs. FIG. 1(b) is a schematic illustration of an arrangement with two reservoirs. FIG. 1(c) is a schematic illustration of the system of FIG. 1(a), with three reservoirs.

FIG. 2 shows images of swabs and threads following a transfer, or attempted transfer, of a charged substance (fluorescein) from a swab (polyurethane) to a thread. FIG. 2(a) shows the swab (left side) and thread (right side) following an attempted transfer without direct contact between the swab and the thread. FIG. 2(b) shows the swab (left side) and thread (right side) following an attempted transfer without any applied electric field while the swab and the thread were in direct contact. FIG. 2(c) shows the swab (left side) and thread (right side) following a transfer completed with direct contact between the swab and the thread.

FIG. 3 is a schematic illustration of an inorganic thread-based device incorporating a cartridge design in accordance with one embodiment of the present disclosure.

FIG. 4 is a schematic illustration of an inorganic thread-based device incorporating a multiplexed, cartridge design in accordance with another embodiment of the present disclosure.

FIG. 5 shows images demonstrating the transfer of a range of charged substances from a sample applicator (swab) to a thread using electrophoresis. In FIG. 5(a), the left-hand image shows a fluorescent image of the swab after the transfer is effected by electrophoresis, and the right-hand image (thread line) shows the positioning of the fluorescent fluorescein on the thread post-transfer and post-electrophoretic focusing. FIG. 5(b) shows the swab for the alkaloid composition post-transfer (left-half image) and the right-half image shows the alkaloids on the thread post-transfer and post-focusing. FIG. 5(c) shows the swab for the labelled protein post-transfer on the left, and the thread post-transfer on the right showing the presence of the protein.

FIG. 6 shows images demonstrating the transfer of a charged substance, fluorescein, to threads of different materials including (a) nylon, (b) mercerised cotton, (c) cotton and (d) polyester. In each sub-figure, a fluorescent image of the swab after the transfer is shown on the left and a fluorescent image of the transferred and focussed analyte band on the thread is shown on the right.

FIG. 7 shows images demonstrating the transfer of a charged substance (in particular, fluorescein) from different swab materials, (a) polyurethane and (b) cotton, onto a thread using electrophoresis. In each sub-figure, a fluorescent image of the swab after the transfer is shown on the left and a fluorescent image of the transferred and focussed analyte band on the thread is shown on the right.

FIG. 8 is a schematic illustration of a reservoir design for the sample loading reservoir.

FIG. 9 shows images of the swab and thread junctions before (shown on left) and after (shown on right) the transfer of a charged substance (fluorescein) from a swab (polyurethane) to a thread in the absence of any additional liquid in the sample transfer reservoir.

FIG. 10 shows images of the swab and thread junctions before (shown on left) and after (shown on right) the transfer of a charged substance (fluorescein) from a swab (polyurethane) to a thread in the absence of any additional liquid in the sample transfer reservoir. FIG. 10(a) shows images of the swab and thread junction for the transfer of a liquid sample from a pre-wetted swab. FIG. 10(b) shows images of the swab and thread junction for the transfer of a liquid sample from a dry swab. FIG. 10(c) shows images of the swab and thread junction for the transfer of a powder sample from a dry swab.

FIG. 11 shows images of the swab and thread junctions before (shown on left) and after (shown on right) the transfer of a charged substance (fluorescein) from a swab (polyurethane) to a thread. FIG. 11(a) shows images of the swab and thread junction for the transfer of a liquid sample dried on a plastic surface and swabbed with a pre-wetted swab. FIG. 11(b) shows images of the swab and thread junction for the transfer of a liquid sample dried on a plastic surface and swabbed with a dry swab. FIG. 11(c) shows images of the swab and thread junction for the transfer of a liquid sample dried on a metallic surface and swabbed with a pre-wetted swab. FIG. 11(d) shows images of the swab and thread junction for the transfer of a liquid sample dried on a metallic surface and swabbed with a dry swab. FIG. 11(e) shows images of the swab and thread junction for the transfer of a liquid sample dried on a wooden surface and swabbed with a pre-wetted swab.

FIG. 12 shows images of the swab and thread junction before (shown on left) and after (shown on right) the transfer of a charged substance (fluorescein) from a swab (polyurethane) to a thread from spiked saliva samples in the presence of a cell lysis buffer.

FIG. 13 shows images of the transfer, separation, and concentration of a charged substance (fluorescently tagged DNA) present in a complex sample (defibrillated sheep blood) from a swab (polyurethane) to a thread. FIG. 13(a) shows the image of the swabbed spiked blood sample. FIG. 13(b) shows the image of the separated blood cells and haemoglobin on the thread. FIG. 13(c) shows the microscopic image of the lysed cells on the thread. FIG. 13(d) shows the image of the focussed fluorescently tagged DNA band on the thread.

FIG. 14 shows images for splitting a charged substance (fluorescein) transferred from a swab (polyurethane) onto two threads each with a discrete operational reservoir, in accordance with one embodiment of the present disclosure. FIG. 14(a) shows the arrangement of the splitting threads with their individual operational reservoirs. FIG. 14(b) shows the image of the swab in direct contact with a single thread. FIG. 14(c) shows the image of the fluorescent analyte split onto two threads.

FIG. 15 shows images of the inorganic filaments, thread and yarn. FIG. 15(a) shows a microscope image of a silica thread (strand) showing the presence of silica glass filaments (10 um diameter). FIG. 15(b) is a photograph of a silica glass thread (strand). FIG. 15(c) is a photograph of a twisted or braided silica yarn. FIG. 15(d) is a microscope image of a twisted or braided silica yarn.

FIG. 16 shows images of the inorganic filaments, thread or yarn. FIG. 16(a) shows a microscope image of a twisted Shin-Etsu inorganic yarn. FIG. 16(b) shows a microscope image of a Quartzel inorganic yarn. FIG. 16(c) shows a microscope image of a Shin-Etsu single inorganic strand yarn. FIG. 16(d) shows a microscope image of a hand braided inorganic Shin-Etsu yarn of 30 tex at 4× objective magnification.

FIG. 17 shows images of the inorganic filaments, thread or yarn. FIG. 17(a) is a photograph of a hand braided Shin-Etsu yarn with a total tex of 30. FIG. 17(b) is a photograph of a self braided Shin Etsu yarn with a total tex of 300.

FIG. 18 shows an image of strip of quartz glass cloth and the woven structure of the strip and the arrangement of individual quartz/silica capillaries within the strip.

FIG. 19 shows images of an analyte transfer experiment using woven inorganic strips having different configurations. FIG. 19(a) is a time series of electrophoretic movement of fluorescein on a 1 cm wide woven silica strip. FIG. 19(b) is a time series of electrophoretic movement of fluorescein on a 1 cm silica strip having thinner edges than the strip shown in FIG. 19(a).

FIG. 20 shows images of electrophoresis being performed to separate a variety of charged substances on inorganic yarns. The charged substance appears in the photographs as a concentrated fluorescein band after isotachophoresis on a) Shin-Etsu twisted yarn, b) Quartzel twisted yarn and c) hand braided single Shin Etsu yarn of 30 tex.

FIG. 21 shows images of the analyte transfer experiment using inorganic yarn. FIG. 19(a) shows a photograph of a reservoir with a polyurethane swab at the end of the transfer experiment. FIG. 19(b) is a photograph showing a concentrated band of fluorescein on silica yarn, illustrating transfer of the analyte from the swab to the yarn.

FIG. 22 shows a band of fluorescein obtained after electrophoretic focusing on a silica yarn. FIG. 22(a) shows the band visualized at 0.5 ppb fluorescein. FIG. 22(b) shows the band visualized at 1 ppb fluorescein.

FIG. 23 shows a band of fluorescein (at 0.5 ppb and 1 ppb) obtained after electrophoretic focusing on a nylon yarn. FIG. 23(a) shows the band visualized at 0.5 ppb fluorescein. FIG. 23(b) shows the band visualized at 1 ppb fluorescein.

FIG. 24 shows signal-to-noise for the band of fluorescein concentrated on a silica yarn. FIG. 24(a) shows the signal-to-noise analysis at 0.5 ppb fluorescein. FIG. 24(b) shows the signal-to-noise analysis at 0.1 ppb fluorescein.

FIG. 25 shows signal-to-noise analysis for the band of fluorescein concentrated on a nylon thread. FIG. 25(a) shows the signal-to-noise analysis at 0.5 ppb fluorescein. FIG. 25(b) shows the signal-to-noise analysis at 0.1 ppb fluorescein.

FIG. 26 shows the results of a direct transfer of a low concentration of fluorescein from a polyurethane swab to a silica yarn. FIG. 26(a) shows an image of the polyurethane swab with 0.5 ppb solution of fluorescein before the start of transfer experiment. FIG. 26(b) shows an image of the concentrated fluorescein band on silica yarn after the transfer and isotachophoresis experiment. FIG. 26(c) is a graph of fluorescent intensity vs distance graph showing focussing of the fluorescein into a thin band.

FIG. 27 shows fluorescent images of a silica yarn before and during the direct transfer experiment of FIG. 26. FIG. 27(a) shows the silica yarn before the start of the experiment. FIGS. 27(b)-(f) shows the concentrated fluorescein band at the respective positions of 1 cm, 1.5 cm, 2 cm, 2.5 cm, and 3 cm along the silica yarn.

FIG. 28 shows microscopic images of different yarn types at 4× magnification at the conclusion of an electrophoresis experiment. FIG. 28(a) shows a twisted Shin-Etsu yarn. FIG. 28(b) shows a hand braided yarn of 30 tex.

### DETAILED DESCRIPTION OF PREFERRED EMBODIMENTS

As described above, the present disclosure provides a range of methods, devices, systems and cartridges that are reliant on an inorganic thread for providing the substrate or matrix for performing electrophoresis to separate charged substances.

The present disclosure provides a method for separating charged substances by electrophoresis, comprising:
providing a length of an inorganic thread;
contacting an electrolyte with an outer surface of the inorganic thread so as to cause wetting of the outer surface of the inorganic thread (i.e., coating an electrolyte on the thread),
loading the charged substances into the electrolyte wetting the outer surface of the inorganic thread in the region of a loading zone portion of the inorganic thread; and
applying an electric field across the length of inorganic thread to cause the charged substances to be separated along the inorganic thread under the influence of the electric field.

Expressed in alternate terms, the method comprises:
providing a length of an inorganic thread extending between a pair of electrodes;
coating an electrolyte on the thread so as to create a continuous liquid bridge between the two electrodes,
loading the charged substances onto the inorganic thread; and
applying an electric field across the length of inorganic thread between the electrodes to cause the charged substances to be separated along the inorganic thread under the influence of the electric field.

### Inorganic Thread

The thread is one that provides a directional pathway for electro-osmotic flow from one location to another.

A “thread” is a fibre or strand that comprises one or more filaments or fibrils. A filament or fibril is the smallest component of the thread—and typically multiple filaments or fibrils will make up a single “thread”. In some embodiments, the inorganic thread comprises at least 5 filaments or fibrils. In some embodiments the inorganic thread comprises at least 10 filaments or fibrils, at least 20, at least 50, or at least 100 filaments or fibrils. A single thread (comprising the above filaments or fibrils) can then be converted into a yarn—which is made up of two or more such threads or strands.

The thread of the present disclosure may be used in singular form, or in the form of a yarn. In some embodiments the thread is a single thread. In some embodiments, a yarn is used. A yarn may generally comprise any number of threads. In some embodiments, the yarn comprises between 1 and 5 inorganic threads or between 3 and 5 inorganic threads. In some embodiments the yarn comprises at least 2 inorganic threads, at least 3 inorganic threads, or at least 5 inorganic threads or at least 10 inorganic threads or at least 15 inorganic threads. In some embodiments the yarn comprises up to 20 inorganic threads; for example, the yarn may comprise between 1 and 20 inorganic threads, or between 2 and 20, 3 and 20, 10 and 20 inorganic threads, etc. In embodiments where the yarn comprises between 5 and 20 inorganic threads, preferably the threads are tightly braided as this may provide for better loading capacity of a charged substance transferred from, e.g., a sample applicator and/or electrolyte solution to the inorganic thread and/or better separation efficiency during electrophoresis.

In other words, a thread may comprise a plurality of filaments or fibrils and a yarn may comprise a plurality of threads. Both threads and yarns are applicable to the methods and uses described herein and are encompassed in a “thread”. The inorganic threads of the yarn, or filaments or fibrils of the thread, may be aligned parallel to one another, or coiled, wound, braided, twisted, piled, spun, bundled or intertwined. A thread (or yarn) so-arranged may be referred to as “structured”. Structured threads (or yarn) may, in use, may improves wetting of the inorganic thread (or yarn) compared to unstructured inorganic thread (or yarn). For example, structured inorganic thread may provide increased surface area, greater surface roughness (e.g., complexity), larger electrolyte holding capacity, and/or higher resolution of a charged substance than its unstructured counterpart. Whatever the case, a thread provides a directional pathway for electro-osmotic flow from one location to another; i.e., is unidirectional.

A thread (or yarn) is distinguished from a “fabric”. A fabric provides a pathway for electro-osmotic flow from one location to more than one other location, i.e., they tend to be bi- or multi-directional. That is, a fabric tends to combine threads or yarns into an arrangement that includes other than parallel alignment, for example a cross-hatched weave allowing multi-directional diffusion in the fabric in all directions of the weave.

The thread of the present disclosure is an inorganic thread. This means that the thread is formed form an inorganic material. The term “inorganic” in this case refers to materials that are not based on carbon or hydrocarbon-like substances. Examples of materials outside the scope of “inorganic” are natural fibres such as cotton and silk, polymeric substances based on carbon and hydrogen (and optionally oxygen and nitrogen) such as nylon, polyester and so forth. The inorganic materials are suitably based on silica or comprise silica. The inorganic threads are preferably at least 80% by weight silica based. This allows for some surface functionalisation of the inorganic thread. The inorganic thread may, for example, be a silica thread or a glass fibre thread. In other embodiments, the inorganic thread may be formed from one or more materials selected from the group consisting of: silica, silicon dioxide, silanol, quartz, aluminium dioxide, magnesium oxide, calcium oxide, and combinations thereof.

The inorganic thread may have a hydrophilic outer surface. The inorganic thread may comprise outer surface silanol groups. Alternatively, the outer surface may be surface-modified or coated to provide different outer surface functional groups. By way of example, the inorganic thread may comprise a graphene oxide outer surface. This may be provided by a thread made up of one or more filaments or fibrils, one or more or each of which having a hydrophilic outer surface.

As explained above, the inorganic thread is “open” to the environment, so a sample can be loaded at any point as required along the thread and can be accessed or contacted as required at any point downstream from where it is loaded along the thread. The “outer” surface is the surface exposed to the environment, i.e., outward facing, and which may be accessed or contacted. As the outer surface is important for access or contacting, it is the surface of focus herein, though description of properties relating to the outer surface may apply equally to surfaces which may be found in a thread which are not exposed, for example inward facing surfaces of filaments of fibrils making up the thread.

The inorganic thread may generally be of any length and diameter. The thread is suitably formed of solid filaments or fibrils (i.e., without any central bore). While any length or diameter may be used, the diameter may typically be not more than 10 mm in diameter, preferably not more than 9, 8,7, 6, 5, 4, 3, 2 or 1 mm in diameter. The diameter of the filaments or fibrils is preferably between 1 and 50 μm, more preferably between 3-14 μm. The thread diameter is therefore preferably at least about 50 μm or at least 100 μm, or at least 250 μm, or at least 400 μm allowing for multiple filaments or fibrils. The thread diameter may be up to 1000 μm, up to 800 μm or otherwise. Any minimum and maximum value can be combined to form a range—such as 100 μm-1000 μm, 250 μm-800 μm and so forth. If the thread comprises coiled, wound, braided or intertwined multi-thread components, the diameter refers to the total thread diameter. In another arrangement contemplated herein, the thread may be in the form of a multi-thread network, such as a net, or may include multi-thread network section(s) and single-thread section(s), though importantly this remains as distinct from a fabric; a multi-thread network may be regarded as a collection of individual threads as described herein.

Thread dimensions are typically measured in the unit of “tex”, which refers to the weight of 1000 m of the thread. The thread may have a weight of at least 5 tex. Where an inorganic yarn comprising inorganic threads is used, the yarn may have a weight of at least 20 tex. Test work shows that a yarn of 30 tex is effective and based on this a yarn weight of as low as 20 tex is expected to be effective. The tex of a yarn is influenced by the number of inorganic threads or strands in the yarn, and in turn by the number of inorganic filaments or fibrils present in the yarn. It is also influenced by the material from which the inorganic threads are formed (e.g., glass weight compared to other inorganic materials).

The length of the thread may be any length suitable for performing the separation. The length may be at least 15 mm. Separations have been performed on threads as short as 2 cm in length. The thread length may be at least 20 mm or at least 30 mm. In some embodiments the thread length is between 15 mm and 300 mm, or between 20 mm and 300 mm. The upper length may be 250 mm, 200 mm or 15 mm. This length applies to the distance between the points of the inorganic thread across which the electric filed is applied. The distance may be the linear distance between the points of contact with the electrodes (e.g., point of emergence from the reservoirs in which the electrodes are positioned).

The inorganic thread may be a plasma-treated thread. The inorganic thread may be chemically treated to increase hydrophilic properties. The inorganic thread may be functionalised or unfunctionalised. The inorganic thread may be coated or uncoated.

Inorganic thread suitable for use in the present disclosure may not be conventionally available in a form ready for use in the present disclosure. Specifically, silica yarns made available by suppliers to various industries may be provided with a coating on the silica yarn outer surface to avoid “fluff” generation. Accordingly, surface treatment may be required to remove the outer surface coating, to return the surface to the native inorganic material surface.

In the case of a silica thread, the outer surface suitably comprises outer surface silanol groups.

In the case of any inorganic thread material, the inorganic thread outer surface is suitably a hydrophilic surface.

The inorganic thread is suitably one that supports the formation of an electro-fluidic circuit between the pair of electrodes. Expressed another way, the thread allows for a fluid bridge to be formed along the length of the thread between the electrodes.

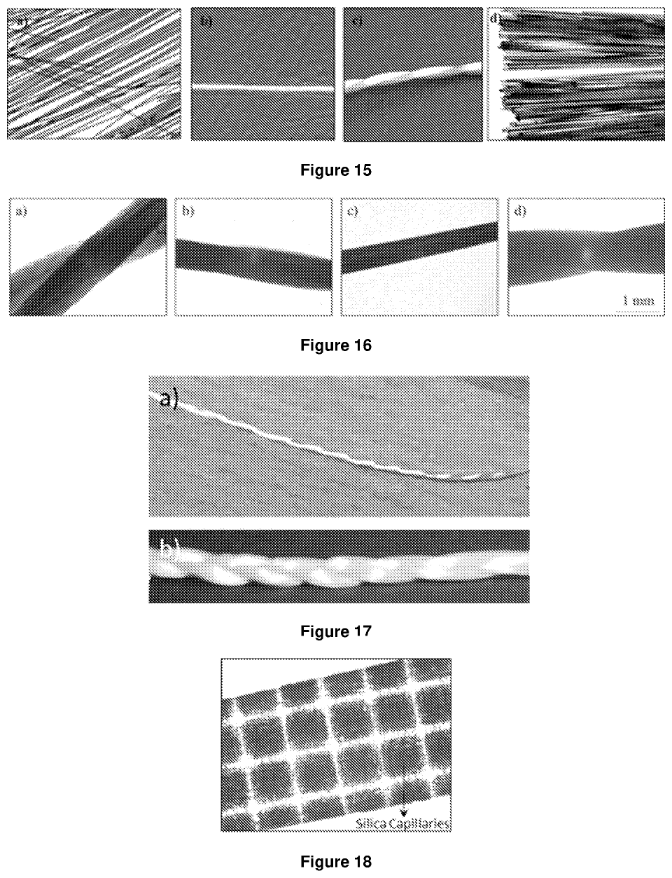

With reference to the Figures, FIG. 15 shows one suitable embodiment for the inorganic thread. FIG. 15(a) demonstrates that the thread is comprised of multiple inorganic glass filaments. These filaments have a diameter between 1-30 μm. A plurality of these fibres may then be bundled together to form threads (FIG. 15b), and a plurality of threads may then be bundled together to form yarns (FIG. 15c). These inorganic threads or yarns may then be further arranged in a woven, twisted, piled, spun, braided or knitted structure, though remaining as distinct from a fabric.

Typically, inorganic thread or yarn such as those made of silica are manufactured from tens to hundreds of silica glass filaments or fibrils bundled together to form the thread or yarn. To reduce the generation of fluff during manufacturing these glass-based textiles, the glass fibres are coated with a number of outer surface coating agents including silane coupling reagents and organic sizing agents. These coatings make the outer surface of the glass fibres hydrophobic. Due to the hydrophobic nature of these glass textiles, it may not be feasible to wet them or form a liquid bridge especially when using a water-based electrolyte, which are important properties for a substrate to be suitable for wetting by the electrolyte and for acting as the electrophoresis matrix (i.e., support matrix for performing electrophoresis).

A number of different inorganic yarns were assessed for their suitability for use as the electrophoresis matrix after plasma treatment. In the case of hydrophobic textiles, the present applicant has advantageously found that plasma treatment of these inorganic yarns by a vacuum plasma reactor removed organic material such as the hydrophobic outer surface coating agents from the glass filaments or fibrils, improving the wetting properties or wettability of the inorganic yarns especially when using a water-based electrolyte. The time period of plasma treatment may be, for example, at least 2 minutes, or at least 4 minutes.

The plasma treatment provides a method of stripping an outer surface coating from the inorganic thread having an outer surface coating.

The vacuum plasma treatment may comprise:
(i) enclosing the inorganic thread in a reaction chamber;
(ii) applying vacuum to evacuate the reaction chamber;
(iii) supplying a gas to the reaction chamber;
(iv) forming a plasma in the reaction chamber and exposing the inorganic thread to the plasma for a time period sufficient to strip (or degrade or break down) the outer surface coating from the inorganic thread.

The time period of exposure of the inorganic thread to the plasma may be one that is sufficient to reduce the water contact angle of the inorganic thread.

The time period of exposure may be between about 1 minute to about 15 minutes.

The plasma treatment is suitably controlled to be sufficient to remove the hydrophobic coating whilst not significantly impacting on the mechanical strength of the thread. Excessive plasma treatment may make the thread brittle. Spectroscopic techniques such as FTIR and Raman spectroscopy can be used to ascertain whether there has been effective removal of the thread outer surface coating.

It was found through the experimental work that plasma treatment of the threads can effectively remove the coatings on outer surface-coated inorganic threads even from within the narrow capillary structures present in these threads to make them suitable for electrophoresis.

The gas used in the plasma treatment may comprise oxygen, nitrogen, nitrous oxide, ammonia, carbon dioxide, air, non-reactive gases such as argon or a combination of two or more thereof.

The gas in the reaction chamber may comprise a mixture of about 90 to about 99% support gas to about 1% to about 10% reactive gas.

The gas pressure in the chamber may suitably be between 0.1 to 0.4 mbar

The gas in the chamber may be supplied at a feed rate of between 10 to 100 Nl/h.

The igniting the plasma in the gas in the reaction chamber may carried out by capacitively coupling RF power to the gas in the reaction chamber. The RF power output may at a frequency in a range of about 1 kHz to about 10 MHz.

The outer surface coating that is removed by plasma treatment comprises coupling agents, bonding agents, organic sizing agents, organic coatings or combinations thereof.

During plasma treatment of the thread, the thread should be positioned to enable a maximum surface area to be exposed to the plasma. After treatment, the plasma-treated threads may be stored in an inert atmosphere prior to use, as the outer surface activity of the threads following plasma treatment may make the thread prone to contamination. Further exemplification of the plasma treatment for an inorganic yard to remove any outer surface coating or outer surface treatment is set out in the Examples.

In some embodiments, the inorganic thread is free of a separate channel-forming substrate. By way of explanation, some techniques in the prior art may rely on channels formed in a block or substrate, into which a thread may be positioned. However, in the present disclosure, the inorganic thread itself forms a pathway for an electrolyte solution and for the passage of charged substances under the application of an electric field.

### Sample Applicator

While many embodiments of the disclosure do not rely on a sample transfer step to the inorganic thread, some embodiments do provide for a sample transfer from a sample applicator to the inorganic thread.

In some such embodiments, the sample applicator may be in the form of a swab. A swab is a pad, piece or sheet of material that is able to be wiped or touched to a surface to effect the transfer of a sample (substances) from the surface to the swab to facilitate collection of the sample. The “material” of the swab may be in the form of a fabric (woven, non-woven, felted or otherwise), in the form of a soft block (e.g., a foamed resilient pad or block of any suitable shape), paper, or otherwise. The swab may further comprise a stick or similar for holding of the swab—e.g., in the manner of a “Q-tip”. The swab may be formed from natural or synthetic material. Notable examples are polyurethane swabs, flocked nylon swabs, and cotton swabs. Synthetic polymer swabs may be preferred, such as polyurethane swabs.

In some embodiments, the method comprises transferring the sample from the sample applicator by immersing the sample applicator into an electrolyte solution and contacting the electrolyte solution with the inorganic thread. In preferred embodiments, the method comprises transferring the sample from the sample applicator to the inorganic thread without an intervening transfer into a solution. The transfer is direct and autonomous from human interaction.

In preferred embodiments, at least 50% of the charged substance is transferred from the sample applicator to the inorganic thread. The amount may be at least 80% of the charged substance/analyte or at least 90% of the charged substance/analyte. The test results shown herein demonstrate that one can transfer substantially all of the charged substance/analyte from the sample applicator to the inorganic thread. Expressed another way, the method is able to effect quantitative transfer of the charged substance/analyte from the sample applicator to the inorganic thread.

The method involves the application of the electric field in the presence of an electrolyte.

The presence of an electrolyte refers to contact of the electrolyte with the inorganic thread. This provides the required electrical pathway for the application of an electric field and the transfer of the charged substance to the inorganic thread under the influence of the electric field. That is, an electrolyte is generally taken to be a substance which contains ions which can be mobilised by application of an electric field. Any electrolytes known in the art may be used. Electrolytes may comprise ionic substances. The electrolyte may be a liquid electrolyte or a gel electrolyte or otherwise. In preferred embodiments, the electrolyte is a liquid, preferably water-based and preferably an aqueous solution.

The electrolyte may comprise a volume of the electrolyte—i.e., a “bulk electrolyte” that wets the inorganic thread, or the electrolyte may be present in a smaller volume that just wets the inorganic thread by coating or wicking of the inorganic thread by the electrolyte. By a “volume” is meant an amount that exceeds the amount required for wetting the inorganic thread. The actual volume of bulk electrolyte will typically depend on the volume of the reservoir containing the bulk electrolyte. The bulk electrolyte in this case may constitute a drop, aliquot or pool of electrolyte in the reservoir. The reservoir may be filled by at least 10%, 20%, 30%, 40%, 50%, 60% or at least 70% of its volume by the electrolyte. The amount of electrolyte may be, for example, at least 0.1 mL, at least 0.5 mL, at least 1 mL, at least 5 mL or at least 10 mL. It has surprisingly found that, where the inorganic thread is in a bulk electrolyte, rather than diffusing into the bulk electrolyte to be spread throughout at low concentration, the charged substance follows the pathway of the electric field and transfers from the sample applicator directly to the inorganic thread. Wetting of the inorganic thread by the electrolyte (e.g., by wetting or wicking of the matrix with electrolyte) similarly enables the transfer of a high percentage of the charged substance from the sample applicator onto the inorganic thread.

An electrophoretic process is performed on the charged substance on the inorganic thread. There is the option to use the one electrolyte composition that is used to wet the inorganic thread throughout the process. In the alternative, a combination of electrolytes may be used, particularly where it is desired to perform an isotachophoretic separation of charged substances/analytes after transfer of the analyte to the inorganic thread. In that case, in some embodiments, the electrolyte used in the sample transfer reservoir is a terminating electrolyte. A terminating electrolyte may alternatively be referred to as a trailing electrolyte. The term is well understood in the art of the disclosure. The choice of terminating electrolyte may depend on the charged substance or analyte to be subjected to the transfer. Further, if focusing or concentration of the charged substance, or a target substance among the charged substances, is to be performed, then the selection of the terminating electrolyte may be impacted by the identity of the target substance and/or the leading electrolyte. The combination of a terminating electrolyte and a leading electrolyte impacts on the isotachophoretic separation or concentration of the relevant analyte(s).

Where the charged substance is transferred to the inorganic thread through the application of the electric field in the presence of the electrolyte; it is found that a higher proportion of the charged substance transfers to the electrophoresis matrix than is transferred into the electrolyte (e.g., the bulk electrolyte). The charged substance can be transferred from the sample applicator to the inorganic thread by immersing the sample applicator into an electrolyte solution and contacting the electrolyte solution with the inorganic thread.

Preferably, the charged substance is transferred directly from the sample applicator to the inorganic thread, without a separate transfer into the bulk electrolyte and subsequently out of the bulk electrolyte and onto the inorganic thread. The electric field passes through the inorganic thread, and thread in particular, providing an electric field pathway for the transfer of the charged substances from the sample applicator directly to the thread, rather than the substance transferring into the bulk electrolyte. The inorganic thread provides a focused exit route for the charged substances away from the sample applicator. It is found that the highest proportion of the charged substance transfers to the electrophoresis matrix in this way.

Any type of sample that contains a charged substance or substances may be used as the sample. Examples include biological samples, pharmaceuticals, environmental samples (e.g., soil), laboratory chemical analyses samples and so forth. One type of sample that may suitably be subjected to the method is a biological sample. Examples of suitable biological samples that may be subjected to the method include saliva, blood, cells, cell lining and mucous. Another type of sample that may suitably be subjected to the method is a laboratory chemical analysis sample. Examples of suitable laboratory chemical analysis sample that may be subjected to the method are numerous, and include essentially any sample containing a charged substance.

The term “charged substance” refers to a substance that has a charged state or is polarised in the relevant conditions such that it can move under the influence of an electric field. As an example, the charged substance may be a substance that is ionisable in water. The charged substance may be a charged pharmaceutical, a charged analyte (e.g., a possible contaminating species in an environmental material), or a charged biomolecule, among other examples. In one embodiment, the charged substance may be a charged biomolecule. The charged biomolecule may be selected from polynucleotides, polypeptides, proteins or various combinations thereof. The biomolecules may be natural or synthetic. Polynucleotides may comprise of DNA, RNA or a combination of DNA and RNA. The polynucleotides may be single stranded or double stranded. Double stranded polynucleotides are those in which all the bases are paired with a complementary base on a second polynucleotide strand. For example, some of the single stranded polynucleotides may comprise a sequence complementary to other single stranded polynucleotides. The polynucleotides may also have a combination of single and double stranded portions wherein only a subset of the bases are engaged in complementary base-pairing.

The polynucleotides may comprise of 10 to 1000 nucleotides. For example, the polynucleotides may comprise 10 to 50 nucleotides, 10 to 100 nucleotides, 10 to 250 nucleotides, 10 to 500 nucleotides, 100 to 1000 nucleotides, 250 to 1000 nucleotides, or 500 to 1000 nucleotides.

In another embodiment, the charged substance may be a charged chemical analysis species. Examples are numerous, and include for example a compound having a charged functional group such as an amine, carboxylic acid, carbocation, carbanion, phosphate, sulphate, sulfoxide, sulfonium, nitro, transition metal complexes and so forth.

In some embodiments, the method comprises:
subjecting the charged substance to electrophoresis to separate a target analyte that forms a component of the charged substance (or constitutes the charged substance) into a focused band on the inorganic thread.

The above method allows for the clean-up of a test analyte from a complex material, such as a complex biological mixture. The clean-up of the test analyte may, where desired, allow for direct analysis to be performed on the inorganic thread as it passes through a detection zone of the electrophoretic matrix (e.g., thread).

In some embodiments, the separated target analyte is concentrated on the inorganic thread through the electrophoretic process. The specific form of electrophoresis may be isotachophoresis.

The location of concentration, or the time-period taken for the concentrated target analyte to reach a particular location, enables the target analyte to be separated from other components in the charged substance. This also allows for concentration of a particular charged substance (or target analyte) to be concentrated in one location on the inorganic thread. As the inorganic thread is an “open system”, the thread can be divided at the required location to separate the concentrated region of target analyte. Otherwise, if the location of sample transfer is performed at a sample transfer zone of the thread, then at a spaced location from the sample transfer zone, analysis can be performed on the thread to detect for the target analyte. The zone at which this detection is performed may be described as a detection zone. The detection may be of any suitable type, such as PCR analysis, microanalytical techniques or otherwise. The detection step may involve RNA amplification if required, according to any process known in the art, including reverse-transcription loop-mediated isothermal amplification (RT-LAMP).

The method described above may further comprise the step of:
detecting for the presence of the target analyte.

For methods performed on biological samples, the method may comprise: contacting the sample applicator containing the sample with a lysis buffer to lyse the cells present in the biological sample. The lysis buffer suitably also contains electrolyte components for the subsequent electrophoretic separation. If the method involves an isotachophoretic separation, the electrolyte may be a terminating electrolyte.

Further details of the method for performing the charged substance transfer will be described in further detail below with reference to the figures and examples. Additional features of the method will become apparent with reference to the discussion of a system for performing the method. Those system features also provide an indication of preferred features of the method.

Also described herein is a system for the transfer of a charged substance from a sample on a sample applicator to an inorganic thread, the system comprising components including:
a sample transfer reservoir;
an inorganic thread positioned through the sample transfer reservoir;
a pair of electrodes positioned to enable the application of an electric field across the inorganic thread;
   wherein the components are positioned such that the sample applicator contacts the inorganic thread during the application of an electric field along the inorganic thread to enable transfer of the charged substance from the sample applicator to the inorganic thread.

Also provided is a system for the transfer of a charged substance from a sample on a sample applicator to an inorganic thread, the system comprising components including:
a receiver for receiving the sample applicator;
a sample transfer reservoir;
an inorganic thread positioned through the sample transfer reservoir;
a pair of electrodes positioned to enable the application of an electric field across the thread;
   wherein the components are positioned such that the sample applicator contacts the inorganic thread during the application of an electric field across the thread to enable transfer of the charged substance from the sample applicator to the inorganic thread.

It is noted that the sample transfer reservoir may serve as the receiver for receiving the sample applicator, or the receiver may be in the form of a separate feature of the device into which the swab is positioned, before it is moved into contact with the inorganic thread. The receiver may be actuated between one position in which the sample applicator is received by the device, and a second position where the sample applicator is positioned in contact with the inorganic thread.

It is also noted that the system or device may be in the form of a cartridge. Alternatively, the system may include a cartridge that provides one or more of the components of the system described above. Further details of this cartridge-type arrangement and other possible arrangements are described below.

In some embodiments, the thread has a first end and a second end, and the electrodes are positioned one towards each end of the thread.

In some embodiments, the system comprises an operation reservoir spaced apart from the sample transfer reservoir, and the inorganic thread spans the transfer reservoir and the operation reservoir. The inorganic thread traverses each of the transfer reservoir and the operation reservoir, and there between. The operation reservoir is a reservoir at which an operation is performed on the charged substance. The operation may be a chemical reaction involving the charged substance, or detection of the charged substance or otherwise. Accordingly, the operation reservoir may be alternatively referred to as a chemical reaction reservoir or a detection reservoir, or otherwise, depending on the operation being performed.

In some embodiments, the system comprises two electrolyte reservoirs. One electrolyte reservoir is positioned to one end of the thread, and the second electrolyte reservoir is positioned to the other end of the thread. Each reservoir is for receiving electrolyte. The electrolytes may be the same. Alternatively, the electrolytes may be different. For an isotachophoretic process, one electrolyte may be a terminating (or trailing) electrolyte and the other may be a leading electrolyte.

The electrodes present as components of the system may be positioned one in each of the two electrolyte reservoirs. The electrodes may be denoted as a positive electrode and a negative electrode, respectively, although the polarity depends on the voltage potential applied across the electrodes.

The reservoirs in each instance are suitably able to hold liquid, such as a liquid electrolyte. In preferred embodiments, the sample loading and operation reservoirs need to allow for bulk liquid electrolyte, or other liquid reagents, to be held, to facilitate charged substance loading and operations to be performed on the charged substance, respectively.

In some embodiments, the system comprises an array of components for performing a plurality of operations (such as detections/analyses/chemical reactions) contemporaneously on one or more samples. The array may comprise at least two sequences, each sequence containing:
at least three reservoirs each,
an inorganic thread extending between the reservoirs, and
a pair of electrodes, one in each of the reservoirs to each end of the thread.

In some embodiments, there are at least four reservoirs for each sequence. These may include, for instance, a sample transfer reservoir and an operation reservoir, in addition to the reservoirs at each end of the threads which are associated with the electrodes.

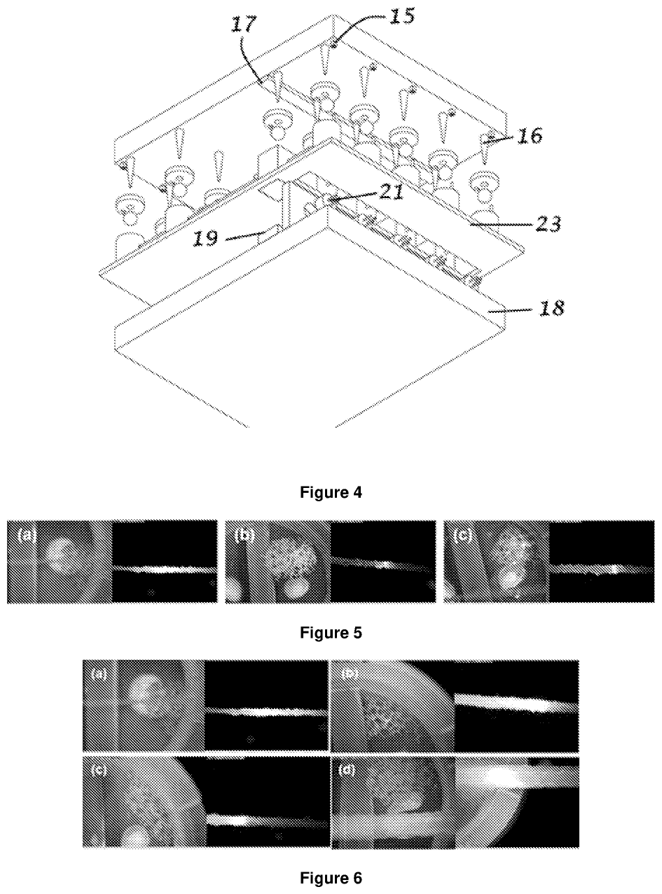

The first and last reservoirs in each sequence may be shared between the sequences. That is, the same positive and negative electrodes (and associated reservoirs) may be shared between the sequences. Thus, each thread for each sequence may start in a shared, single reservoir at one end, and terminate in a shared, single reservoir at the opposite ends of the threads. The central reservoir (or reservoirs if more than one) positioned along the inorganic thread between each of the end reservoirs are separate for each inorganic thread (sequence) in the array. Alternatively, each sequence may comprise separate reservoirs and electrodes at each end of each inorganic thread. (Such an arrangement is illustrated in FIG. 4.) The system may further comprise a controller for controlling the application of a voltage potential across the inorganic thread, and performing any other steps such as electrophoretic separation and analysis.

It is noted that the system may comprise a cartridge that provides each of the features (i) to (iv) indicated above for the system. Thus, the cartridge may comprise a series of reservoirs (e.g., three, four or more reservoirs), a pair of electrodes, an inorganic thread and reagent pods. In alternative arrangements, the cartridge may comprise a series of at least four reservoirs, an inorganic thread connecting the reservoirs in series and reagent pods containing reagents suitable for each reservoir. One or more of the reservoirs may further comprise a magnetic bead for stirring contents in the reservoir. A magnetic bead may be positioned in an operation reservoir, as one example. A magnetic bead may additionally, or alternatively, be positioned in the sample transfer reservoir, if required for stirring the contents of that reservoir. As described in further detail below, the reagents in the reagent pods may include the required electrolyte, lysis buffer or reagents for performing a chemical reaction or aiding analysis as required in the associated reservoir.

Also developed by the applicant, which can be used in combination with the sample-transfer system or independently of the sample-transfer system, is a system that allows for one or more operations to be performed on the charged substance taken from the sample.

In one example, there is provided an electrophoresis system for performing an operation on a charged substance, the system comprising:
a first electrolyte reservoir comprising a first electrode;
a second electrolyte reservoir comprising a second electrode;
an inorganic thread extending between the first and second electrolyte reservoirs;
a sample loading reservoir positioned along the inorganic thread between the first and second electrolyte reservoirs for loading sample onto the inorganic thread;
a controller for controlling the application of an electric field across the inorganic thread so as to effect movement of the charged substance along the thread from the sample loading reservoir towards the second electrolyte reservoir; and
a operation zone positioned along the thread between the sample loading reservoir and the second electrolyte reservoir, at which an operation can be performed involving the charged substance.

As noted above, the system may include just one of the reservoirs selected from the sample loading reservoir and the operation reservoir, but in embodiments described below both reservoirs are described. The system may alternatively comprise just two electrolyte reservoirs, and no sample loading reservoir or operation reservoir—instead, there may be a sample loading zone and/or an operation zone (e.g., a detection zone). The description should be read in light of this.

The operation zone is preferably a zone of the inorganic thread that is in the region of an operation reservoir. The operation reservoir is a reservoir that receives a liquid, such as an electrolyte and/or reagent, in the vicinity of which an operation can be performed on the charged substance. Examples of “operations” being performed involving the charged substance include analysis of the charged substance, coupling of the charged substance to a marker, a chemical reaction or a transformation involving the charged substance, and so forth. The sample loading reservoir is free of any electrode, as is the operation reservoir (when present).

The system may comprise further electrode-free reservoirs. Such reservoirs may enable the performance of more than one operation along the flow-path of the charged substance along the inorganic thread.

The system, or components of the system, may be provided in a cartridge format. In one example where the system includes four (or more) reservoirs, the cartridge for use in an electrophoresis instrument comprises:
a first electrolyte reservoir comprising a first electrode;
a second electrolyte reservoir comprising a second electrode;
an inorganic thread extending between the first and second electrolyte reservoirs;
a sample loading reservoir positioned along the thread between the first and second electrolyte reservoirs for loading sample onto the inorganic thread; and
a operation reservoir positioned along the thread between the sample loading reservoir and the second electrolyte reservoir within which an operation can be performed involving the charged substance.

The cartridge may further comprise electrolyte for each of the first electrolyte reservoir and the second electrolyte reservoir. The cartridge may additionally comprise electrically conductive reagent for the sample loading reservoir and the operation reservoir.

In some embodiments, the system includes an array comprising multiple inorganic threads, at least one each of the first and second electrolyte reservoirs and first and second electrodes, and multiple sets of said sample loading reservoirs and operation reservoirs. If provided in cartridge form, each cartridge may be for a single set, or a single cartridge may contain multiple sets of the reservoirs, electrodes and inorganic threads.

An array of such components allows for multiple parallel runs to be performed to achieve multiplexed or high throughput analysis. These may be performed on either a single sample (or single sample applicator/swab) or form multiple samples (sample applicators/swabs). Multiplexed analysis has been performed by splitting a single inorganic thread into multiple pathways, where each pathway was used to determine a specific marker to provide a more holistic sample analysis and minimise the false positive and negative results that are often obtained when a single marker is analysed. High throughput analysis has been performed by recruiting multiple inorganic threads, substantially in parallel, in which each inorganic thread was used to perform analysis on an individual swab, minimising the average sample analysis time in situations such as epidemics and pandemics.

As indicated above, microfluidic textile analytical devices have been restricted to the use of different electrophoresis matrices (i.e., not inorganic threads). They have also been restricted to electrode-coupled initial and terminating reservoirs. However, the above-described configuration has been developed that allows the use of multiple electrode-free reservoirs, facilitating multi-step analysis and minimising the risks of electrode fouling. Additional reservoirs can be arranged between the initial and terminating electrodes such that they do not break the electro-fluidic circuit while also allowing independent activities, such as sample introduction, concentration, modification, detection, selective uptake or release, etc. The developed system can facilitate the use of microfluidic textile analytical devices in performing complex analytical procedures, which are often required in real-life settings. Moreover, since microfluidic textile analytical devices use high voltages, the availability of electrode free reservoirs for sample manipulation would also promote the generation of safer microfluidic textile analytical devices by preventing user exposure to the live electrodes. The system of this embodiment also enables the user to easily perform an operation on a charged substance taken from a sample.

Also described herein is a method for performing an operation on a charged substance. In some embodiments, the method comprises:
providing a first electrolyte reservoir comprising a first electrode, a second electrolyte reservoir comprising a second electrode and an inorganic thread that extends between the first and second electrolyte reservoirs;
loading a charged substance onto the inorganic thread in a sample loading reservoir positioned along the inorganic thread between the first and second electrolyte reservoirs; and
moving the charged substance along the inorganic thread by electrophoresis from the sample loading reservoir towards an operation zone positioned downstream of the sample loading reservoir in the flow direction of the charged substance by applying an electric filed across the inorganic thread between the first and second electrodes; and
performing an operation involving the charged substance in the operation zone.

The thread may extend through an operation reservoir in which the operation zone of the inorganic thread is positioned, and the operation may be performed in the operation reservoir. There may be more than one such operation reservoir (or operation zones) traversed by the thread, and allowing for different operations to be performed in each of said reservoirs (or in each of said zones). The movement of the charged substance along the inorganic thread involves the application of an electric field across the thread.

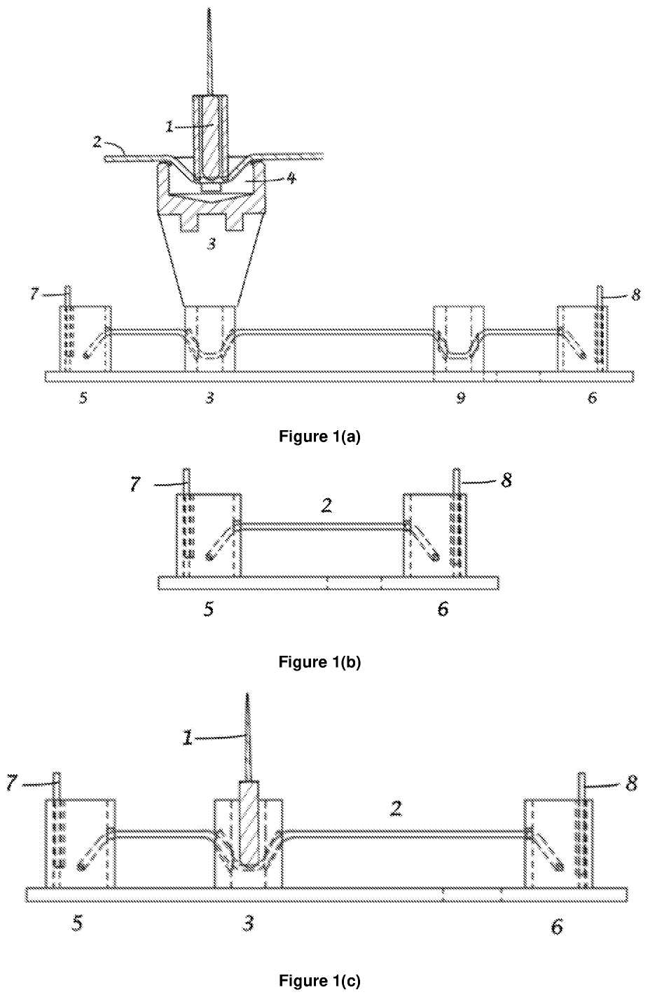

A suitable set-up for performing one or more of the above operations is shown in FIG. 1, parts (a) to (c). In FIG. 1(a), components of the system are illustrated, including the following:
1. Swab
2. Inorganic thread
3. Sample transfer reservoir
4. Electrolyte
5. First electrolyte reservoir
6. Second electrolyte reservoir
7. First electrode
8. Second electrode
9. Operation reservoir

The system includes first and second electrolyte reservoirs (5, 6) each containing a first and second electrode, respectively. The first electrode is negatively charged, and the second electrode is positively charged through the application of an electric potential across the electrodes. The circuit is completed by the electrolytes that wet the thread (2). Any suitable electrolyte (or combination of electrolytes) can be used. In one example, the electrolytes include a terminating electrolyte which is in the first electrolyte reservoir (5) and the leading electrolyte which is in the second electrolyte reservoir (6). The sample transfer reservoir (3) also contains electrolyte, as does the operation reservoir (9). The thread is wetted by the electrolyte. The coating may be in any suitable state, such as liquid or gel, and therefore the thread may in an alternative example be coated by a conductive hydrogel coating.

A swab of a suitable material such as polyurethane or otherwise, is contacted with a surface to take a sample from the surface. The surface could be a part of a human such as the hand, mouth, tongue or otherwise, or the surface may be an inanimate surface such as an environmental sample including soil, water body, flora and the like, or a laboratory apparatus including a petri dish, reaction vessel and the like. The swab (1) containing the sample (the sample comprising any number of components including one or more charged substances), is brought into direct contact with the thread (2) in the sample transfer reservoir (3). While in contact with the thread, an electric field (voltage potential) is applied across the electrodes (7,8), for a time period to effect transfer of charged substances from the swab to the thread.

FIG. 1(b) shows the same system, but without any sample transfer reservoir or operation reservoir. FIG. 1(c) shows the same system again, but without an operation reservoir. The reference numerals are the same as used in FIG. 1(a).

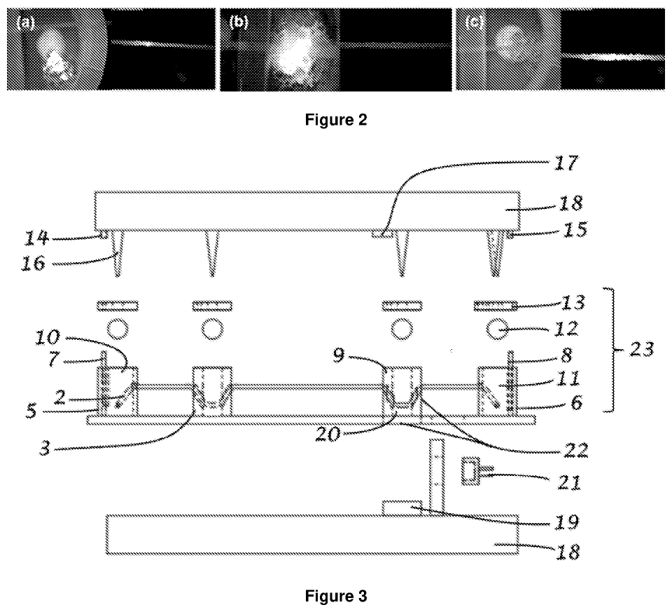

FIG. 2 shows a comparative test showing the impact that direct contact and applied electric field has on the transfer, compared to when the thread and swab are not contacted or the electric field is not applied. In the comparative test of FIG. 2, the threads used were organic threads, but the work was subsequently repeated with an inorganic thread. When the thread and swab are not contacted or the electric field is not applied the transfer occurs via the liquid medium (being an electrolyte) or through diffusion. In the top left image of FIG. 2(a), a swab is shown following an attempted transfer of a fluorescent analyte, where the transfer took place by applying an electric filed across the thread, in the presence of an electrolyte, without direct contact between the swab and the thread. This resulted in only 6±3% of analyte transfer. The right-hand image of FIG. 2(a) shows the thread, where only a weak fluorescent band of the analyte was observed in line with its limited transfer. Similarly, FIG. 2(b) shows the swab (left side) and thread (right side) after an attempted transfer where the swab and the thread were in direct contact, however, an electric field was not applied across the thread. This resulted in only 5±1% of analyte transfer. In contrast, FIG. 2(c) shows the swab (left side) and thread (right side) following a transfer completed with direct contact between the swab and the thread while an electric field was applied across the thread. This time, a transfer of 94±1% was observed from the swab onto the thread.

Referring to FIG. 1, with the swab (1) still in contact with the thread (2), or following removal of the swab (1), the electric field is applied (or continues to be applied) to effect movement of the charged substance along the thread. Different charged substances (if more than one is present) move at different rates in view of their differing electroosmotic and electrophoretic mobilities. This effects separation of different charged substances (if multiple charged substances are present) and/or concentration of charged substances along the thread. The time period for a target charged substance, or analyte, to pass along the thread to the operation reservoir will be known for known substances, and thus the charged substance can be controlled to be positioned in the operation reservoir at a known time period for detection by a suitable detector mechanism, or for reaction, or otherwise.

By way of one example, the qualitative and quantitative analysis of the number and type of viral colonies present in the bodily fluids, such as cell lining, saliva, mucous, and blood can be performed using a combined approach of isotachophoresis (ITP) and reverse transcription loop-mediated isothermal amplification (RT-LAMP).

ITP facilitates the extraction and concentration of the nucleic acid content, and RT-LAMP facilitates selective amplification of the desired RNA for real-time quantitation.

Thread based ITP is used to perform sample clean-up, extraction, and pre-concentration of viral RNA. The user may position the collected swab into the second well, being the sample transfer reservoir (3) which may be viewed as a RNA extraction and lysis well, where the nucleic acid from the sample on the swab transfers directly to the thread (2) (otherwise referred to as a fibre). The absorbed RNA would then be focussed into the third well, being the operation reservoir (9), using ITP.

RT-LAMP is an isothermal DNA amplification technique, which circumvents the need for cumbersome PCR instruments and is usually performed in simple Eppendorf vials by subjecting them to a constant temperature (usually 50-70° C.). Like PCR, the DNA amplification during RT-LAMP can be monitored using fluorescent tagging dyes, such as SYBR Green I (SG), which binds to DNA during its amplification and hence allows its easy quantification. Moreover, RT-LAMP technique only requires 15-20 minutes of analysis time as compared to more than 2 hours required for PCR, and the former also allows the use of multiple primers to provide high selectivity for the desired RNA strand.

In one example, a cartridge-type system may be used. An example of one cartridge-type system is shown in FIG. 3. In FIG. 3, the same numerals are used to denote the same features as in FIG. 1, with the additional reference numerals indicating as follows:
10. Terminating electrolyte
11. Leading electrolyte
12. Reagent pods
13. Caps
14. First electrode socket
15. Second electrode socket
16. Retractable needles
17. Heater
18. Electronic chamber
19. Light source
20. RT-LAMP cuvette
21. Photodiode
22. Transparent windows

Each sample analysis cartridge consists of four wells (5, 3, 9, 6), two electrodes (7, 8), a thread (2), and four reagent pods (12). The cartridge body may be formed from plastic, with an inorganic thread, and metal electrodes (e.g., stainless steel) so that new cartridge can be used for each analysis to prevent any cross-contamination and hence false-positive results.

By positioning the electrodes in separate wells (5, 6), ITP can be used to focus RNA or other analytes on the inorganic thread.

Magnetic beads (not shown) may be pre-packaged into the second well (3) to allow stirring with a rotating magnet in the bottom panel of the electronic chamber (18), to assist with quick desorption and lysing. It is anticipated that magnetic beads are not specifically required in this well, but this is nevertheless an option. RNA would be focused in the third well, being the operation reservoir (9), where RT-LAMP would be performed using the overhead heater (17). The high speed of RT-LAMP would be improved due to pre-concentration of the RNA and its presence on a high surface-area to volume ratio thread. The DNA would bind with the available SYBR Green I (SG) fluorescent dye, and its quantity analysed using the lower light source (19) and individual photodiodes.

Precise quantities of the required reagents/electrolytes can be delivered in pre-packaged pods (12) within their respective wells. The pods may be pierced using retractable needles (16) to release the contents prior to the analysis.

FIG. 4 illustrates an array containing similar elements to those shown in FIG. 3. This arrangement contains an electronic chamber (18) which forms a part of the fixed instrument part of the system. There is a disposable cartridge (23) which contains an array of four wells and inorganic threads extending between the sets of four wells. The cartridge is designed for plug-and-play assembly with the fixed instrument.

Multiplexing enables analysis of multiple samples within the same total analysis time of 15-20 minutes. This enables mass testing especially at screening points such as airports, where samples can be collected as soon as passengers depart from the plane and their results would be available by the time they go through the immigration process.

Multiplexing can also be used to perform multi-stage analysis to identify all the positive cases, where one row could be used to screen for all SARS virus, the second row could be used to identify RdRP gene, and the third row could be used to perform a discriminatory test, as recommended.

### EXAMPLES

The present disclosure will now be described in further detail with reference to the accompanying non-limiting examples, which demonstrate the efficacy of the disclosure.

It is noted that Examples 1 to 10 were performed using an organic thread, but the work was repeated on inorganic threads to demonstrate the efficacy of the processes and procedures with inorganic threads—with demonstrated transfer of the charged substances to the inorganic thread, wetting and wicking of the thread, and successful separations and detections of the charged substances on the inorganic thread. Examples 1 to 10 are presented for background information and full understanding of the disclosure. Examples using an inorganic thread are provided in Examples 11 to 17.

### Example 1—Transfer of a Variety of Charged Substances From Swab to Thread Using Electrophoresis

Tests were conducted to show the efficacy of transfer of a variety of charged organic molecules of varying sizes from a swab to the nylon thread.

### Reagents and Materials

Tris-(hydroxyl methyl) amino-methane (TRIS), 4-(2-hydroxyethyl)-1-piperazineethanesulfonic acid (HEPES), hydrochloric acid, fluorescein sodium salt, coptisine chloride, palmatine chloride, and myoglobin, were obtained from Sigma-Aldrich (New South Wales, Australia). Berberine chloride, European Pharmacopoeia Reference Standard was purchased from EQDM Council of Europe (France). Chromeo 488 NHS-Ester was purchased from Santa Cruz Biotechnology (Santa Cruz, CA, USA). OraSwab plain was purchased from Confident Care products (New South Wales, Australia). Solutions were prepared in water from a Milli-Q Water Plus system from Millipore (Bedford, MA, USA), with a resistivity of 18.2 MΩcm.

100% nylon (diameter (Ø) 803±53 μm, woolly nylon stretches overlocking thread, QA Thread, China) was used for the thread, otherwise referred to as the fibre-based microfluidic. Later examples involved the use of alternative thread materials. Threads (“fibres”) were washed in Milli-Q water and sonicated for 10 minutes 3 times to remove impurities on the outer surface. Subsequently, fibres were plasma treated by a vacuum plasma reactor (K1050X plasma asher (Quorum Emitech, UK)) to increase the wicking property, facilitating the sample application.

### Device Fabrication

Buffer reservoirs and a Lego type platform were designed with Fusion360 CAD software (Autodesk) and printed using an Eden 260VS (Stratasys, MN, USA) with the VeroClear build material, and SUP707 water-soluble support. The support material was cleaned with water and 2% NaOH as required. Subsequently, the reservoirs were rinsed and soaked in Milli-Q water for a day. The reservoirs were reused multiple times following a wash with water and 2% NaOH. Each reservoir consisted of a bridge to guide and submerge the thread in the buffer and two legs to mount them in the platform. The first and second reservoirs also consisted of a slot to hold the required electrode and a Lego type lock to adjust the thread's position and tension.

The assembly procedure involved two steps. Firstly, buffer reservoirs were inserted into the base according to the desired format and length. Secondly, the thread (fibre) was tensioned between the buffer reservoirs. In all experiments, fibres were kept wet with leading electrolyte solution during the electrophoresis process. The direction of electrophoretic migration was dictated by the analyte's charged state and the polarity of the electrodes in the first and second reservoirs.

The same device fabrication was later followed for preparing equivalent devices with inorganic threads in place of the nylon threads.

Fluorescence images were obtained using a USB microscope AM4113T-GFBW (Dino-Lite Premier, Clarkson, WA, Australia) equipped with a blue light-emitting diode for excitation and a 510 nm emission filter. The microscope was controlled using DinoCapture 2.0 software. Fluorescence intensities of images and videos were processed with Image J software for the quantification of target fluorescent analytes.

A high voltage power supply was used for the introduction of voltage for all the fibre-based ITP experiments. For negatively charged analytes, such as fluorescein and myoglobin, the experiments were carried out in anodic mode (cathode in the inlet and anode in the outlet buffer reservoirs), and vice-versa for the positively charged analytes, such as alkaloids. The system was controlled using a 12-Bit, 10 KS/s multifunction DAQ system (USB-6008 OEM, National Instruments, Austin, TX, USA).

### Fibre-Based Isotachophoresis (ITP) Operation

In the case of fluorescein and myoglobin, a 5 mM TRIS/2.5 mM HEPES solution was used as the terminating electrolyte (TE), and 20 mM TRIS/10 mM HCl solution was used as the leading electrolyte (LE). While in the case of alkaloids, a 20 mM β-alanine solution was used as TE and 20 mM potassium acetate solution was used as the LE. The electroosmotic flow was suppressed by adding 0.1% PVP to the TE and LE solutions. After assembling the fibre-based ITP setup and placing the electrodes, 500 μL of TE and LE were added to the first and second reservoirs, respectively. The fibres were then wetted with the LE. The samples were swabbed form their 2 μL droplets of the desired concentration, which were previously spread over a glass slide. Before applying the voltage, the system was equilibrated for 1 minute to reduce the capillary action along the fibre. Finally, constant voltage or current was applied to initiate the ITP procedures.

### Results

Molecules that were transferred from the swab to the nylon thread through direct contact of the swab to the nylon thread, in the presence of an electrolyte and through the application of a voltage potential across the thread included fluorescein, alkaloids (in particular, a mixture of coptisine, palmatine, berberine) and protein (chromeo 488 NHS ester-labelled myoglobin). The results are shown in FIG. 5. In FIG. 5(a), the left-hand image shows a fluorescent image of the swab after the transfer is effected by electrophoresis, and the right-hand image (thread line) shows the positioning of the fluorescent fluorescein on the thread post-transfer and post-electrophoretic focusing. FIG. 5(b) shows the swab for the alkaloid composition post-transfer (left-half image) and the right-half image shows the alkaloids on the thread post-transfer and post-focusing. FIG. 5(c) shows the swab for the labelled protein post-transfer on the left, and the thread post-transfer on the right showing the presence of the protein. A sample volume of 2 μL was swabbed; the concentration of the analytes ranged from 10-100 ppm.

The molecular weights of these analytes range from 300 g/mol to 17000 g/mol. An instantaneous transfer was observed for all three analytes, and they were further focussed on the thread (in a concentrated band) using isotachophoresis within 2-3 minutes.

An estimate of the percentage transfer was made based on the fluorescence in microscopic images. The images suggest that when the swab is in direct contact with the thread, this results in 30 and 40 times higher recovery for fluorescein and alkaloids, respectively, as compared to when the swab was present isolated in the same buffer reservoir (indirect contact). No observable transfer of myoglobin was observed during indirect contact. In the case of direct contact, a transfer of more than 90% was observed for all three analytes.

### Example 2—Transfer of a Charged Substance From a Swab to Different Thread Materials Using Electrophoresis

Tests were conducted to show the efficacy of transfer of fluorescein, as an exemplary charged substance, to threads of different organic thread materials, including nylon, mercerised cotton, cotton and polyester. Examples 12-17 were later performed with inorganic thread materials to demonstrate efficacy with this thread material. The results with the inorganic thread materials are shown in FIG. 6—(a) is nylon, (b) is mercerised cotton, (c) is cotton (not mercerised) and (d) is polyester. In each sub-figure, a fluorescent image of the swab after the transfer has been shown on the left and a fluorescent image of the transferred and focussed analyte band on the thread is shown on the right. A sample volume of 2 μL was swabbed; the concentration of fluorescein was 10 ppm. Hydrophilic yarns, such as mercerised cotton and cotton, do not require any pre-treatment before their use, whereas, hydrophobic threads, such as nylon and polyester, benefit from pre-treatment to impart wettability to the thread. Hence, untreated mercerised cotton and cotton were used. Nylon was treated with air plasma. The treatment time was 90 seconds. Un-treated hydrophobic thread, polyester (as shown in FIG. 6(d)) resulted in lower analyte mobility, however, still complete transfer of the analyte was observed. Quantitative analysis of the transfer suggested 94±1%, 97±2%, 98±1%, and 80±7% transfer onto nylon, mercerised cotton, cotton, and polyester, respectively.

### Example 3—Transfer of a Charged Substance From Different Swab Materials Onto a Thread Using Electrophoresis

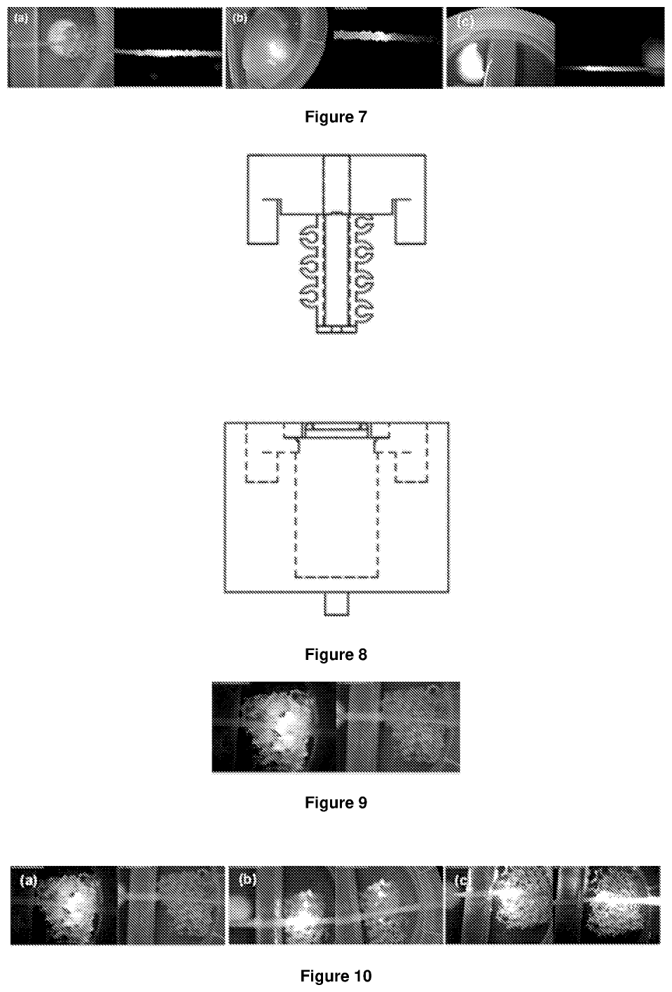

Tests were conducted to show the efficacy of transfer of fluorescein, as an exemplary charged substance, from different swab materials, onto nylon (organic) thread. The three tested swab materials were polyurethane, cotton, and flocked nylon. The results are shown in FIG. 7 where (a) is polyurethane, (b) is cotton, and (c) is flocked nylon. In each sub-figure, a fluorescent image of the swab after the transfer has been shown on the left and a fluorescent image of the transferred and focussed analyte band on the thread is shown on the right. A sample volume of 2 μL was swabbed; the concentration of fluorescein was 10 ppm. A transfer of 94±1% was observed from the polyurethane swab. A transfer amount of 47±7% of fluorescein was observed from the cotton swab. A transfer of 97±2% was observed from the flocked nylon swab. The high fluorescence observed in the flocked nylon swab after transfer was due to the high background fluorescence of the swab itself and not the remaining sample. The limited transfer from the cotton swab is presumably due to an interaction between fluorescein with cotton.

### Example 4—Modified Sample Transfer Reservoir Design

In FIG. 1(a), one of the sample transfer reservoir is shown. In FIG. 8, an alternative design is illustrated. In the arrangement shown in FIG. 8, there is shown a lower section and an upper section. The lower section includes the reservoir opening, with two bores for receiving two corresponding pins located on the upper section. The upper section includes a central plunger with a central slot and seven clips for receiving a thread. The swab may be inserted into the plunger, and the thread wrapped in a spiral fashion through the clips and around the swab. The design allows for greater contact between the thread and the swab, which aids in the transfer of charged substance from the swab to the thread, and for the swab to be moved independently of the reservoir, allowing greater operational freedom.

### Example 5—Transfer of a Charged Substance in the Absence of Any Additional Liquid in the Sample Transfer Reservoir

Tests were conducted to show the efficacy of transfer of fluorescein (as an exemplary charged substance) from polyurethane swab (as an exemplary swab material) onto nylon (organic) thread in the absence of any liquid (buffer, electrolyte or otherwise) in the sample transfer reservoir while the swab was still in direct contact with the thread. While there was no bulk liquid (electrolyte, buffer or otherwise) present, the nylon thread was wetted by leading electrolyte (as described in the “Reagents and Materials” section above), providing the electroresis matrix (thread) with the required presence of electrolyte. The results are shown in FIG. 9 where a fluorescent image of the swab and thread junction before starting the transfer is shown on the left, and a fluorescent image of the swab and thread junction at the end of the transfer experiment is shown on the right. A sample volume of 2 μL was swabbed; the concentration of fluorescein was 10 ppm. A transfer of 97±2% was observed, which is similar to the transfer observed in the presence of a buffer in the sample transfer reservoir. This further confirms the direct transfer of charged analytes from a swab onto a thread (in the presence of electrolyte) and simplifies the system by omitting the need for any additional medium (such as bulk liquid electrolyte) in the sample transfer zone. The same test was later performed on an inorganic thread material (braided silica threads prepared in accordance with Example 11), and a similarly high transfer rate was observed.

### Example 6—Transfer of a Charged Substance From Different Sample and Swab States in the Absence of Any Additional Liquid in the Sample Transfer Reservoir

Tests were conducted to show the efficacy of transfer of fluorescein (as an exemplary charged substance) from polyurethane swab (as an exemplary swab material) onto nylon (organic) thread in the absence of any liquid in the sample transfer reservoir. Different combinations of sample and swab states were studied, i.e., (a) liquid sample swabbed with a swab pre-wetted with the terminating electrolyte (the default state), (b) liquid sample swabbed with a dry swab, and (c) powder sample swabbed with a dry swab. The results are shown in FIG. 10, where (a) is a liquid sample with a pre-wetted swab, (b) is a liquid sample with a dry swab, and (c) is a powder sample with a dry swab. In each combination for this example, the nylon thread was wetted by leading electrolyte (electrolyte as described in the “Reagents and Materials” section above), providing the electrophoresis matrix (thread) with the required electrolyte to support the transfer, and the electric field was applied across the thread to effect the transfer. In each sub-figure, a fluorescent image of the swab and thread junction before starting the transfer is shown on the left and a fluorescent image of the swab and thread junction at the end of the transfer experiment is shown on the right. In the case of the liquid samples, a sample volume of 2 μL was swabbed with a fluorescein concentration of 10 ppm. In the case of the powder samples, 1 mg sample was swabbed. A transfer of 97±2% was observed when a liquid sample on a pre-wetted swab was in direct contact with the thread. A transfer of 93±1% was observed when a liquid sample on a dry swab was in direct contact with the thread. A significant transfer was observed even when a powder sample on a dry swab was in direct contact with the thread. The transfer in the case of the powder sample swabbed with a dry swab in the absence of any bulk liquid medium (but in the presence of liquid electrolyte wetting the thread only) suggests high robustness and wide applicability of the system since it minimises any pre-conditioning of either the sample, swab, or the sample transfer zone. The transfer is expected to occur (without being limited by the theoretical considerations) due to the solvation of the analyte molecules as they encounter the liquid medium (electrolyte) on the thread. The solvated molecules are then electrokinetically transferred from the swab onto the thread.

### Example 7—Transfer of a Charged Substance When Dried Liquid Samples are Swabbed From Different Materials With Different Swab States

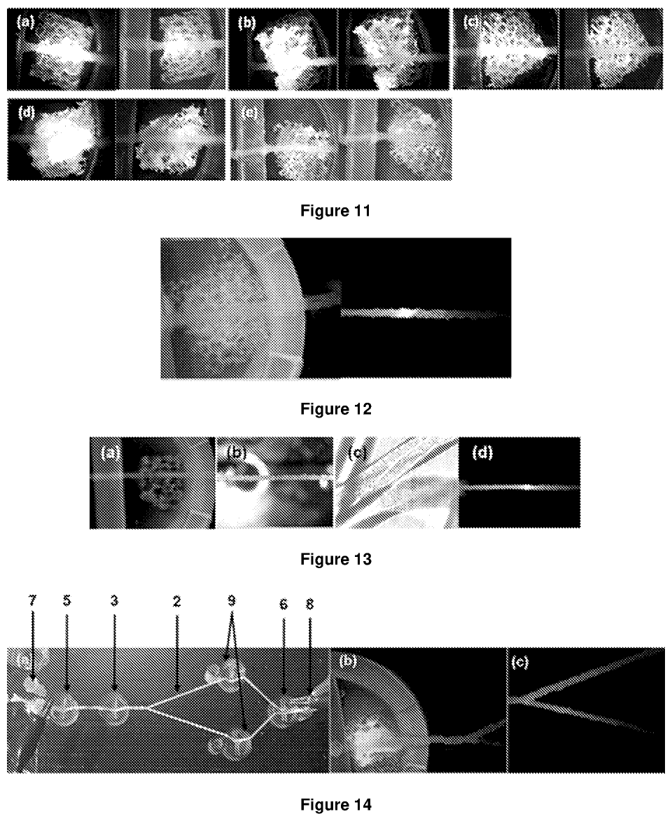

Tests were conducted to show the efficacy of transfer of fluorescein (as an exemplary charged substance) when its dried liquid sample was swabbed with a polyurethane swab (as an exemplary swab) from the surfaces of different types of material, onto a nylon (organic) thread. Three different materials, i.e., (a) plastic, (b) metal, and (c) wood, were swabbed with two different hydrated states of the swab, i.e., (a) pre-wet state and (b) dry state. The results are shown in FIG. 11, where (a) is a plastic surface swabbed with a pre-wetted swab, (b) is a plastic surface swabbed with a dry swab, (c) is a metallic surface swabbed with a pre-wetted swab, (d) is a metallic surface swabbed with a dry swab, and (e) is a wooden surface swabbed with a pre-wetted swab. In each sub-figure, a fluorescent image of the swab and thread junction before starting the transfer is shown on the left and a fluorescent image of the swab and thread junction at the end of the transfer experiment is shown on the right. A sample volume of 10 μL with a fluorescein concentration of 100 ppm was dried on each material. A transfer of more than 90% was observed in each case based on the fluorescent microscope images. In most cases, a pre-wet swab resulted in better sample collection, primarily due to the collected sample's dried nature. However, no significant difference was observed in the transfer of the collected charged analyte from the dry or pre-wet states of the swabs.

### Example 8—Transfer of a Charged Substance in the Presence of a Complex Sample Matrix

Tests were conducted to show the efficacy of transfer of fluorescein (as an exemplary charged substance) in the presence of different sample matrices, from a polyurethane swab (as an exemplary swab), onto a nylon (organic) thread. The results are shown in FIG. 12, where a fluorescent image of the swab and thread junction before starting the transfer from spiked saliva samples in the presence of a cell lysis buffer is shown on the left and a fluorescent image of the swab and thread junction at the end of the transfer experiment is shown on the right. A sample volume of 2 μL was swabbed; the concentration of fluorescein was 10 ppm. A transfer of 95±2% was observed in the presence of the cell lysis buffer. A transfer of 92±3% was observed in the presence of saliva and the cell lysis buffer. The percentage transfer did not show any significant difference in the presence or absence of a complex sample matrix. However, the rate of transfer was lower in the presence of a sample matrix compared to the absence of any sample matrix.

### Example 9—Transfer, Separation, and Concentration of a Charged Substance From a Complex Sample

Tests were conducted to show the efficacy of transfer, separation, and concentration of fluorescently tagged DNA (as an exemplary charged substance) from defibrillated sheep blood (as an exemplary complex sample) using a polyurethane swab (as an exemplary swab) onto a nylon (organic) thread. The results are shown in FIG. 13, where (a) is the image of the swabbed spiked blood sample, (b) is the image of the separated blood cells and haemoglobin on the thread, (c) is the microscopic image of the lysed cells on the thread, and (d) is the fluorescent image of the focussed fluorescently tagged DNA band on the thread. The blood sample was spiked with 20% v/v 1 nm fluorescently tagged DNA, and a sample volume of 10 μL was swabbed from a glass slide. The sample transfer reservoir was filled with the background electrolyte and a cell lysis buffer. The fluorescently tagged DNA was successfully transferred from the swabbed sample, separated from the other blood components, and focussed into a thin band on the thread. Also, the blood cells were successfully lysed using the cell lysis buffer, confirming the ability to modify the swabbed sample simultaneously during the transfer.

### Example 10—Splitting the Transferred Substance Into Multiple Threads Downstream for Multiplexed Analysis

Tests were conducted to show the efficacy of transfer and splitting of fluorescein (as an exemplary charged substance) sample from a single polyurethane swab (as an exemplary swab material) onto two nylon threads. The results are shown in FIG. 14, where (a) is the arrangement of the split threads with their individual operational reservoirs, (b) is the image of the swab in direct contact with a single thread, and (c) is the fluorescent image of the analyte split onto two threads. A sample volume of 2 μL was swabbed; the concentration of fluorescein was 10 ppm. The fluorescent analyte was found to split between two different threads as 50±3% on each. This allows multiple independent analyses along different threads on the same sample. The same device set-up is applicable for inorganic thread materials, based on the test work subsequently completed.

### Example 11—Production of an Inorganic Thread Suitable for Use as an Electrophoresis Matrix

Silica-based yarns were sourced from Shin-Etsu Chemical (SQYG15DT-02, 330 tex, twisted yarn; SQYG 150-02, 10 tex thread) and Saint-Gobain (Quartzel yarn, C9 33×4 S150, 150 tex, twisted yarns). The threads of the yarns were comprised of multiple glass filaments with a diameter between 3 and 14 μm (FIG. 15(a)). A plurality of these filaments (or fibrils) were present in each thread of the textile bundled together to form threads (FIG. 15(b)). These silica glass threads were then further piled and twisted together to form twisted silica yarn (FIG. 15(c)). A microscopic image of a twisted silica yarn is also shown (FIG. 15(d)).

FIG. 16 shows microscopic images of four yarns used in the test work. FIG. 16(a) is a microscopic image of a twisted Shin-Etsu yarn, FIG. 16(b) is a microscopic image of the Quartzel yarn, FIG. 16(c) is a microscopic image of a single strand thread of Shin-Etsu yarn, and FIG. 16(d) is a microscopic image of a hand-braided Shin-Etsu yarn of 30 tex at 4× objective magnification (this yarn is discussed further below).

Braiding: Strands of silica thread were hand-braided to form a yarn. Two types of braid were made with the single strand thread. In the first one, 3 strands of the single strand thread were braided together producing a yarn of 30 tex. (FIG. 16(a) and FIG. 17(a)). In the other one, 10 strands of the single strand thread were piled together and then 3 bundles were braided together, producing a braided yarn of 300 tex (FIG. 17(b)).

It was observed that, for such commercially-available silica “glass”-based textiles, to reduce the generation of fluff during manufacturing, the glass filaments or fibrils were coated with silane coupling reagents and organic sizing agents. These coatings make the surface of the glass fibres hydrophobic. Due to the hydrophobic nature of these glass textiles, it was found to be not feasible to wet them or form a liquid bridge, which are the two properties sought for the substrate to act as an electrophoresis matrix. Consequently, the glass yarns were subjected to plasma treatment in a vacuum plasma reactor to remove organic material and improve their wettability.

Plasma treatment: Tests were conducted to identify the period of time required for plasma treatment to achieve sufficient wettability to allow the braided yarns to act as an electrophoresis matrix. It was discovered that the period of time of plasma treatment was dependent on the weight of the yarn in tex. The tex of a yarn is in turn determined by the number of inorganic glass threads or strands and in turn by the number of inorganic filaments or fibrils present in the yarn.

Inorganic threads or yarns were first arranged in a woven, twisted, piled, spun, braided or knitted structure, then subjected to plasma treatment. For a Shin-Etsu woven or twisted yarn (a silica yarn that is coated with a silicone coating) having a total tex of 330 and consisting of 10 bundles of 33 tex single yarn bundled and twisted together (FIG. 20(a)), plasma treatment of 8 minutes was sufficient to make the Shin-Etsu woven yarn suitable to act as an electrophoresis matrix. For a Quartzel (Saint Gobain) woven or twisted silica yarn having a total tex of 133 and consisting of 4 bundles of 33 tex single yarn bundled and twisted together (FIG. 20(b)), plasma treatment of 6 minutes was sufficient to make the Quartzel woven or knitted yarn suitable to act as an electrophoresis matrix, and additionally also for isotachophoresis. Additional experiments were performed on untwisted groupings of threads, based on the Shin-Etsu material, of 10 tex in weight subjected to plasma treatment for 8 minutes. The performance of such threads indicated that there is a preference for twisting or another form of braiding or interweaving of the threads to optimise liquid bridging (or wicking) required for coating of the thread and the completion of the electrical circuit required to perform electrophoresis.

The yarns of FIGS. 17(a) and 17(b) were also plasma treated for 6 and 8 minutes, respectively. Electrophoresis was performed on these hand-braided and plasma-treated yarns (FIG. 20) using the device set-up as shown in FIG. 1(c). Braiding and twisting assisted with the formation of capillary-like channels between individual filaments or fibrils, which helped with wicking (i.e., holding the liquid bridge) and thus allow for electrophoresis.

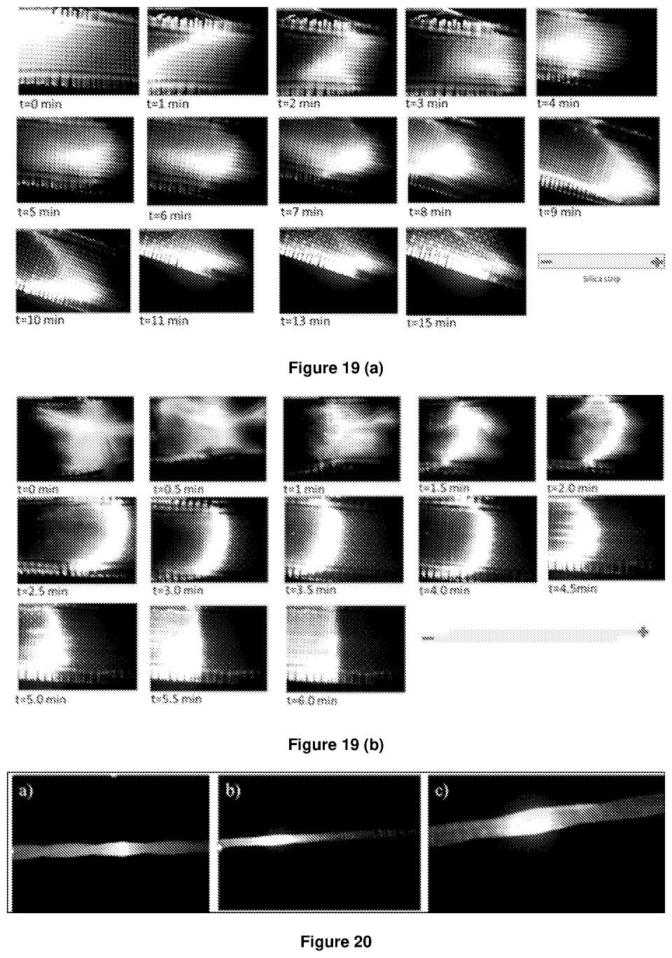

Weaving: The use of woven silica strips as an electrophoresis matrix was also evaluated. The woven structure of the strip and the arrangement of individual quartz/silica capillaries within the strip is shown in FIG. 18.

Two types of strips were tested, one had consistent thickness (Strip 1, FIG. 19(a)) and the other had thinner edges at the opposite ends (Strip 2, FIG. 19(b)). Strip 2 configuration helped to achieve a more uniform distribution of the electric field. In both cases, electrophoretic movement of a model analyte, fluorescein, was observed; however, the analyte band could not be focussed probably due to higher joule heating on the strips as compared to the thread.

### Example 12—Electrophoresis System Design With Plasma Treated Inorganic Silica Yarn

The platform/electrophoresis system consisted of two reservoirs, two electrodes, and silica yarn prepared and plasma treated according to Example 11. Electrodes were placed in both the reservoirs. The distance between the inlet and outlet reservoir was around 8 cm. The inlet and outlet reservoirs were filled with 500 μL terminating (TE) (5 mM TRIS/2.5 mM HEPES with 0.1% PVP) and leading electrolytes (LE) (20 mM TRIS/10 mM HCl with 0.1% PVP), respectively. The silica yarn was wetted with LE before dropping the analyte and applying electric potential. A 2 uL of 1 ppm solution fluorescein was directly dropped 1 cm from the inlet reservoir and then electric potential was applied. The experiments were performed in constant voltage mode with a +900 mV potential applied at the outlet reservoir. Fluorescein moved towards the outlet reservoir and simultaneously concentrated in a thin band.

### Example 13—Transfer of a Variety of Charged Substances From Swab to Inorganic Silica Yarn Using Electrophoresis

For investigating the direct transfer of fluorescein from swab to the silica yarn, the following setup was used. The electrophoresis system comprised three reservoirs to study the analyte transfer from the swab to the thread, as illustrated in FIG. 1(c). The first and third reservoirs housed the electrodes and were filled with 500 μL terminating (TE) (5 mM TRIS/2.5 mM HEPES with 0.1% PVP) and leading electrolytes (LE) (20 mM TRIS/10 mM HCl with 0.1% PVP), respectively. The second well was the sample transfer reservoir which was filled with 500 μL of the TE. The distance between the first and the second reservoir was 3 cm and between the first and the third reservoir was 13 cm. A 2 μL sample of 10 ppm fluorescein was picked up on a polyurethane swab from a glass surface and the swab was placed in the filled sample transfer reservoir.

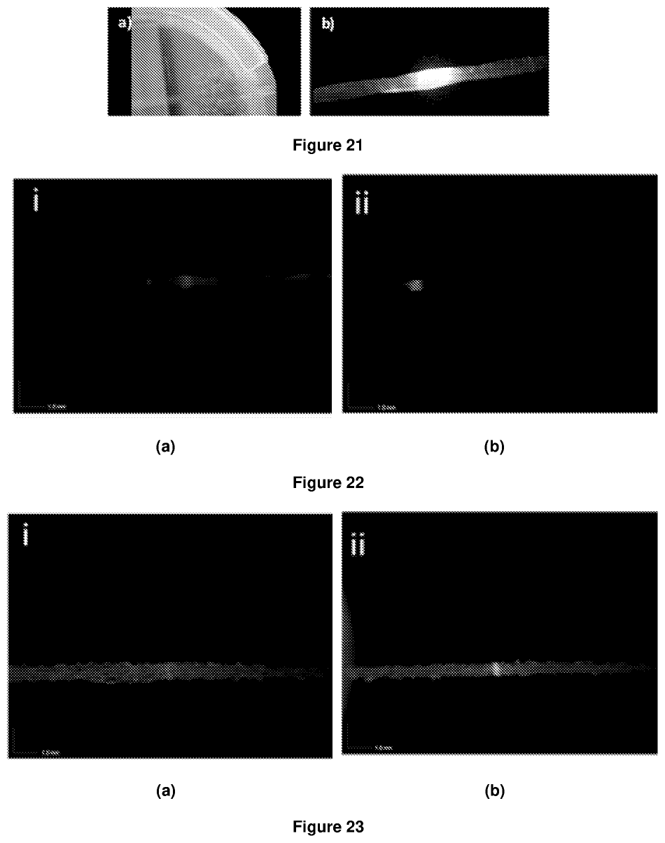

Electrophoresis was performed in constant voltage mode with a +900 mV potential applied at the outlet reservoir. As can be seen in FIG. 21, transfer of fluorescein from the polyurethane swab on to the silica yarn was achieved.

### Example 14—Background Fluorescence Comparative Analysis

Isotachophoresis experiments were performed at different concentrations (0.5 ppb, 1 ppb) on both silica yarn and nylon thread, and the fluorescence signal was plotted as a function of distance to obtain the signal/noise ratios. The experiments were performed according to the following protocol. The platform/electrophoresis system consisted of two reservoirs, electrodes, and silica yarn or nylon thread, as illustrated in FIG. 1(b). Electrodes were placed in both reservoirs. The distance between the inlet and outlet reservoir was around 8 cm. The inlet and outlet reservoirs were filled with 500 μL terminating (TE) (5 mM TRIS/2.5 mM HEPES with 0.1% PVP) and leading electrolytes (LE) (20 mM TRIS/10 mM HCl with 0.1% PVP), respectively. The silica yarn was wetted with LE before dropping the analyte and applying electric potential. A 2 uL of fluorescein sample was directly dropped on the thread at 1 cm from the inlet reservoir, followed by applying the electric potential. The experiments were performed in constant voltage mode with a +0.9 kV potential applied at the outlet reservoirs.

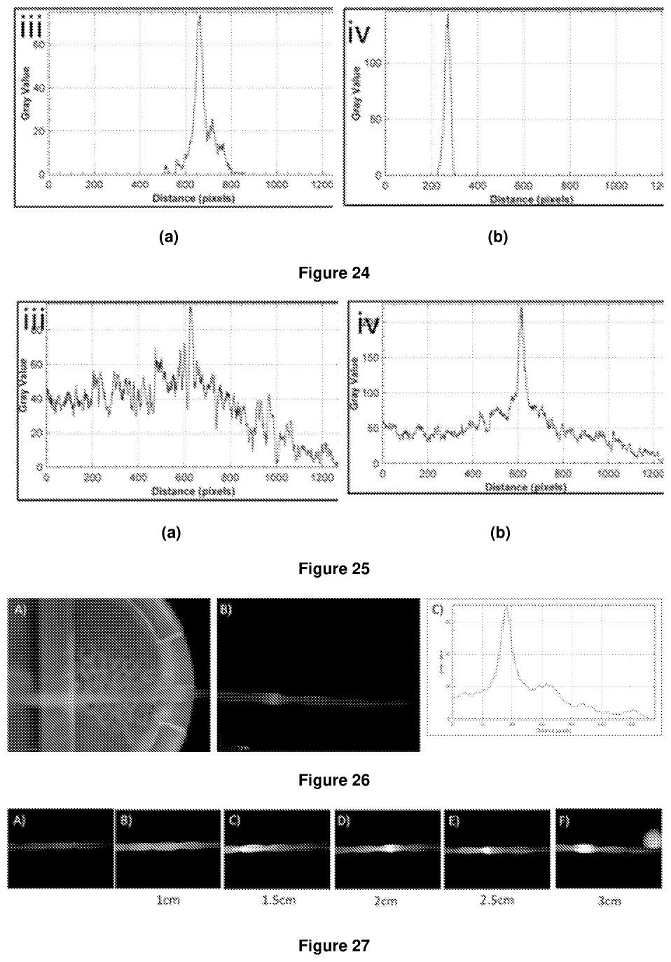

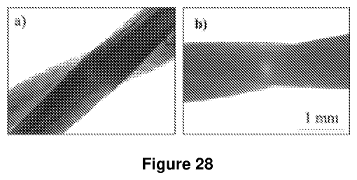

Fluorescein was found to move towards the outlet reservoir and was concentrated in a thin band. The results are shown in FIGS. 22 and 23. FIGS. 22(a) and 22(b) show the concentrated band of fluorescein obtained after electrophoretic focusing of 0.5 ppb and 1 ppb concentrations on a silica yarn. FIGS. 23(a) and 23(b) show the concentrated band of fluorescein obtained after electrophoretic focusing of 0.5 ppb and 1 ppb concentrations on a nylon thread. As can be seen from the fluorescent images, nylon has a higher background fluorescence than silica. The signal-to-noise ratios (S/N) were obtained from the electropherograms. For a concentration of 0.5 ppb and 1 ppb, the S/N for a silica thread is 8 (FIG. 24(a)) and 15 (FIG. 24(b)), respectively, while for a nylon thread, the S/N are 2 (FIG. 25(a)) and 4.4 (FIG. 25(b)), respectively. The higher signal-to-noise ratio on silica yarn compared to nylon thread is due to the low background fluorescence of silica. Thus, a low concentration, like 0.5 ppb, can be easily detected on the silica thread with improved detectability as compared with the nylon thread.

### Example 15—Analysis of a Swab Transfer to an Inorganic Thread

For investigating the direct transfer of low concentration of fluorescein (as an exemplary charged substance) from a polyurethane swab (as an exemplary swab material) to silica yarn, the following setup was used. The electrophoresis system comprised three reservoirs as illustrated in FIG. 1(c) to study the analyte transfer from the swab to the thread. The first and third reservoirs housed the electrodes and were filled with 500 μL terminating (TE) (5 mM TRIS/2.5 mM HEPES with 0.1% PVP) and leading electrolytes (LE) (20 mM TRIS/10 mM HCl with 0.1% PVP), respectively. The second well was the sample injection well, where the swabbed sample was placed and filled with 500 μL of the TE. A 2 μL sample of 0.5 μg/L fluorescein was dropped on the polyurethane swab (equal to 1 pg of fluorescein [1×10-12 g]), and the swab was placed in the sample preparation reservoir. The experiments were performed in constant voltage mode with a +0.9 kV potential applied at the outlet reservoir.

Fluorescein transferred from the swab onto the silica yarn and subsequently moved towards the outlet reservoir and simultaneously concentrated in a thin band (˜3 mm wide), which was very clearly detectable against a very minimal background fluorescence from the silica itself. The results are shown in FIG. 26, where (a) is the image of the swabbed 0.5 ppb solution of fluorescein, (b) is the image of the concentrated fluorescein band on silica yarn, and (c) is graphs the fluorescent intensity vs distance demonstrating the concentration of the fluorescein into a thin band. Imaging this back by intensity, it can be seen in FIG. 26(c), that the peak height is approximately 5-6 times the background fluctuations (noise), indicating an approximate detection limit of 0.1 μg/L based upon a 2 μL sample load onto the swab.

### Example 16—Demonstration of a Peak Migrating Through an Inorganic Thread

The platform/electrophoresis system consisted of two reservoirs and two electrodes, and silica yarn that were plasma treated for 8 minutes. Electrodes were placed in both reservoirs as illustrated in FIG. 1(b). The distance between the inlet and outlet reservoir was around 5 cm. The inlet and outlet reservoirs were filled with 500 μL terminating (TE) (5 mM TRIS/2.5 mM HEPES with 0.1% PVP) and leading electrolytes (LE) (20 mM TRIS/10 mM HCl with 0.1% PVP), respectively. The silica yarn was wetted with LE before dropping the analyte and applying electrical potential. A 2 uL of 10 mg/L solution fluorescein was directly dropped 1 cm from the inlet reservoir, and then electric potential was applied. The experiments were performed in constant voltage mode with a +0.9 kV potential applied at the outlet reservoir.

Migration and simultaneous concentration of fluorescein into a thin band can be seen from the images in FIG. 27. In FIG. 27, (a) is a fluorescent images of the silica yarn before application of electrical potential, and b)-f) are fluorescent images of the silica yarn after application of electrical potential showing a concentrated fluorescein band at positions on the thread corresponding to 1 cm, 1.5 cm, 2 cm, 2.5 cm and 3 cm migration, respectively.

### Example 17—Analysis of the Electrolyte/Buffer Holding Capacity of an Inorganic Thread

At the end of the electrophoresis experiment of Example 16 independently using a Shin-Etsu twisted yarn and a hand braided single Shin Etsu yarn of 30 tex (i.e., as shown in FIGS. 16(a) and (d), respectively), a section of the yarn (5 cm) was cut and placed in an Eppendorf tube. The yarn was centrifuged to extract the liquid in the yarn. The collected section of the thread was introduced into an empty 0.5 mL Eppendorf tube, which was previously impaled such that only the contents of the threads could be transferred out of it and not the actual thread during centrifugation. The impaled tube was placed in a receiving Eppendorf tube (2 mL) and centrifuged at 3000 rcf for 5 min. The amount of liquid in the collection tube at the end of the centrifugation was measured. For the Shin-Etsu yarn (total tex of 330), consisting of 10 bundles of a 33-tex single yarn bundled and twisted together, the buffer holding capacity was 2.4 uL/cm. For a hand-braided yarn with 3 strands of the single strand yarn (10 tex) braided together, producing a yarn of 30 tex, the buffer holding capacity was 0.75 uL/cm.

Various modifications may be made to the embodiments described above with reference to the Figures and Examples without departing from the spirit and scope of the disclosure.

In the present specification and claims, the term “comprising” or variations such as “comprises” or “comprising” is used in an inclusive sense, i.e., to specify the presence of the stated features but not to preclude the presence or addition of further features.

1-85. (canceled)

86. A method for separating charged substances by electrophoresis, comprising:
providing a length of an inorganic thread;
contacting an electrolyte with an outer surface of the inorganic thread so as to cause wetting of the outer surface of the inorganic thread and wicking along the inorganic thread;
loading the charged substances into the electrolyte wetting the outer surface of the inorganic thread in the region of a loading zone portion of the inorganic thread; and
applying an electric field across the length of inorganic thread to cause the charged substances to be separated along the inorganic thread under the influence of the electric field.

87. The method of claim 86, wherein the application of an electric field across the length of the inorganic thread results in separation and concentration of at least one of said charged substances along the inorganic thread, and the method further comprises detecting for the presence of one or more of the charged substances.

88. The method of claim 86, wherein the inorganic thread extends between a first electrolyte reservoir and a second electrolyte reservoir, wherein the electric field is applied across the inorganic thread via a pair of electrodes positioned to apply the electric field along the inorganic thread, and wherein a first electrode of the pair of electrodes is positioned in said first electrolyte reservoir, and the second electrode of the pair of electrodes is positioned in said second electrolyte reservoir, and wherein the inorganic thread is in electrical connection with the first and second electrodes.

89. The method of claim 88, wherein the inorganic thread is wetted by the electrolyte between the two electrolyte reservoirs, and the charged substances are loaded into the electrolyte at a loading zone positioned between the two electrolyte reservoirs, and wherein the application of the electric field effects a transfer of at least one of the charged substances along the inorganic thread away from the loading zone.

90. The method of claim 89, wherein one of said charged substances is a target analyte, and the method further comprises:
subjecting said charged substances transferred along the inorganic thread to electrophoresis to separate the target analyte into a focused band on the inorganic thread; and
detecting for the presence of the target analyte.

91. The method of claim 90, wherein the detection is performed by UV detection or fluorescence without any background fluorescence from the inorganic thread.

92. The method of claim 86, wherein the electrolyte containing the charged substances is on an outer surface of the inorganic thread and is open to the environment to enable access to the charged substances along the length of the inorganic thread, and wherein a portion of the inorganic thread is separated away to isolate that portion of the thread containing a target analyte from the charged substances and the target analyte is recovered from the separated portion of the thread.

93. The method of claim 86, wherein the loading of the charged substances into the electrolyte wetting the inorganic thread comprises bringing a sample applicator containing a sample comprising the charged substances into contact with the inorganic thread at a loading zone portion of the inorganic thread, and applying an electric field across the inorganic thread to load charged substances from the sample onto the inorganic thread.

94. An electrophoresis device for separating charged substances by electrophoresis, comprising:
a length of inorganic thread with an outer surface that is wettable by an electrolyte and allows wicking along the inorganic thread; and
means for applying an electric field along the length of inorganic thread, so that when charged substances are loaded into electrolyte wetting the inorganic thread, the charged substances are separated along the inorganic thread under the influence of the electric field.

95. The electrophoresis device of claim 94, wherein the inorganic thread comprises a yarn formed from a plurality of threads.

96. The electrophoresis device of claim 94, wherein the inorganic thread is formed from one or more materials selected the group consisting of: silica, silicon dioxide, silanol, quartz, aluminum dioxide, magnesium oxide, calcium oxide, and combinations thereof.

97. The electrophoresis device of claim 94, wherein the inorganic thread is plasma-treated.

98. The electrophoresis device of claim 94, wherein the inorganic thread extends between a first electrolyte reservoir and a second electrolyte reservoir, wherein the means for applying an electric field comprises a pair of electrodes positioned to apply the electric field along the length of the inorganic thread, and wherein a first electrode of the pair of electrodes is positioned in said first electrolyte reservoir, and the second electrode of the pair of electrodes is positioned in said second electrolyte reservoir, and wherein the inorganic thread is in electrical connection with the first and second electrodes.

99. The electrophoresis device of claim 94, wherein the inorganic thread comprises:
a loading zone positioned between the first and second electrolyte reservoirs at which a charged substance is transferred into the electrolyte on the inorganic thread in the region of the loading zone; and/or
an operation zone at which, in use, an operation is performed on at least one charged substance separated and focused into a band under the influence of the means for applying the electric field.

100. The electrophoresis device of claim 94, further comprising a detector for detecting for the presence of at least one of the charged substances that, in use, is separated and focused into a band along the inorganic thread, wherein the detector is a UV detector or fluorescence detector, and is positioned to perform the UV or fluorescence detection on the inorganic thread without any background fluorescence from the inorganic thread.

101. A method for the electrophoretic transfer of a charged substance from a sample on a sample applicator to an inorganic thread, the method comprising:
contacting the sample applicator containing a sample including the charged substance with the inorganic thread in the presence of an electrolyte, or
immersing the sample applicator containing a sample including the charged substance into an electrolyte solution, and contacting the electrolyte solution with the inorganic thread; and
applying an electric field along the inorganic thread to effect transfer of the charged substance from the sample applicator and/or electrolyte solution to the inorganic thread.

102. The method of claim 101, wherein the sample applicator is a swab.

103. The method of claim 101, wherein at least 50% of the charged substance is transferred from the sample applicator to the inorganic thread on the application of an electric field.

104. The method of claim 101, further comprising:
subjecting the charged substance transferred onto the inorganic thread to electrophoresis to separate a target analyte that forms a component of the charged substance into a focused band on the inorganic thread; and
recovery of the charged substance from the inorganic thread.

105. The method claim 101, wherein the electric field is applied along the inorganic thread via a pair of electrodes, wherein a first electrode of the pair of electrodes is positioned in a first electrolyte reservoir, and the second electrode of the pair of electrodes is positioned in a second electrolyte reservoir, and wherein the inorganic thread extends between the first and second electrolyte reservoirs and is in electrical connection with the first and second electrodes.
